(19) United States
(12) Patent Application Publication
(10) Pub. No.: US 2026/0199436 A1
(43) Pub. Date: Jul. 16, 2026

(54) SEMAGLUTIDE IN MEDICAL THERAPY INCLUDING WEIGHT MANAGEMENT

(71) Applicant: Novo Nordisk A/S, Bagsvaerd (DK)

(72) Inventors: Rune Viig Overgaard, Koebenhavn (DK); Anders Bjerring Strathe, Farum (DK); Thomas Buch, Ganloese (DK); Kristian Kandler, Koebenhavn (DK)

(21) Appl. No.: 19/312,400

(22) Filed: Aug. 28, 2025

Related U.S. Application Data
(60) Provisional application No. 63/745,930, filed on Jan. 16, 2025.
(60) Provisional application No. 63/751,401, filed on Jan. 30, 2025.

(30) Foreign Application Priority Data
Apr. 11, 2025 (EP) 25170058.9
Jun. 13, 2025 (EP) 25182706.9

(51) Int. Cl. A61K 38/26; A61K 38/22; A61P 3/04

(52) U.S. Cl. CPC A61K 38/26; A61K 38/22; A61P 3/04

(57) ABSTRACT

The present disclosure provides a GLP-1 receptor agonist semaglutide for use in medical therapy including weight management.

### CROSS-REFERENCE TO RELATED APPLICATIONS

This application claims priority to U.S. Provisional Applications 63/745,930, filed Jan. 16, 2025, and 63/751,401, filed Jan. 30, 2025, European Patent Applications 25170058.9, filed Apr. 11, 2025, and 25182706.9, filed Jun. 13, 2025; the contents of which are incorporated herein by reference.

The present invention relates to methods of medical therapy, including weight management such as obesity, comprising the glucagon-like peptide-1 (GLP-1) receptor agonist semaglutide.

### BACKGROUND

Obesity is one of the most significant public health challenges worldwide, has reached epidemic proportions in most countries around the world. Obesity is associated with increased risk of a variety of comorbidities including cardiovascular disease and risk of early death. Moreover, obesity adversely affects physical and mental health and reduces health related quality of life. Obesity is also associated with decreased cardiorespiratory fitness, which also increases the risk of cardiovascular diseases and all-cause mortality.

Semaglutide has been authorised for use in weight management including obesity (Wegovy®) by once weekly subcutaneous administration in many countries with once weekly dosages of initially 0.25 mg followed by dosage escalation every four weeks to 0.5 mg, then to 1.0 mg, then to 1.7 mg and finally to 2.4 mg; the recommended maintenance dosage is 2.4 mg. The glucose-dependent insulinotropic polypeptide (GIP) receptor and GLP-1 receptor agonist tirzepatide has been authorised for use in weight management including obesity (Zepbound®) by once weekly by subcutaneous administration in many countries with initial dosage of 2.5 mg followed by dosage escalation every four weeks in 2.5 mg increments to 5 mg, if needed then to 7.5 mg and then 10 mg, and if needed then to 12.5 mg and then 15 mg; the dosage escalation are to reduce the risk of gastrointestinal adverse reactions and the recommended maintenance dosage is 5 mg, 10 mg or 15 mg.

### DESCRIPTION OF THE DRAWING

FIG. 1 depicts the weight loss observed in subjects co-dosed with cagrilintide and semaglutide. Vertical reference lines represent first and last dosing of cagrilintide and semaglutide.

### SUMMARY

In some embodiments the invention is directed to methods for weight management of a human subject in need thereof comprising subcutaneous administration of about 7.2 mg semaglutide per week. In some embodiments the invention is directed to methods for treating or reducing the risk of one or more selected from the group consisting of (i) cardiovascular (CV) disease, such as reducing the risk of major adverse cardiovascular event comprising CV death, non-fatal myocardial infarction and non-fatal stroke, (ii) metabolic dysfunction-associated steatohepatitis, and (iii) chronic kidney disease in a human subject in need thereof comprising subcutaneous administration of about 7.2 mg semaglutide per week.

### DESCRIPTION

The present disclosure provides a pharmaceutical composition comprising semaglutide or a pharmaceutical acceptable salt thereof.

The present inventors surprisingly found that once-weekly subcutaneous administration of semaglutide 7.2 mg provided an increased benefit risk profile for human subjects. Greater weight loss was obtained without increasing gastrointestinal adverse events including nausea and vomiting to the same degree for once-weekly subcutaneous administration of semaglutide 7.2 mg compared to 2.4 mg. The proportion of subjects discontinuing treatment was similar for once-weekly subcutaneous administration of semaglutide 7.2 mg and semaglutide 2.4 mg as shown in the Examples herein.

The present inventors surprisingly found that dosage escalation directly from once-weekly subcutaneous administration of semaglutide 2.4 mg to 7.2 mg was tolerable. The dosage increase from 2.4 mg to 7.2 mg (3 times) is markedly higher than the dosage escalation step when increasing from one maintenance dosage to the next for an authorised weight management drug comprising a GLP-1 receptor agonist. As used herein the term “maintenance dosage” may refer to dosages authorised for treatment, e.g. following a dosage escalation period. For tirzepatide, the dosage escalation increase to 10 mg is 1.3 times (10/7.5) and to 15 mg is 1.2 times (15/12.5).

Once-weekly subcutaneous administration of semaglutide 7.2 mg provided significantly greater weight loss compared to 2.4 mg as shown in the Examples herein. In particular, a significantly greater proportion of subjects achieved a body weight reduction of at least 15%, at least 20% and at least 25% for once-weekly subcutaneous administration of semaglutide 7.2 mg compared to 2.4 mg as shown in the Examples herein. In addition, a significantly greater proportion of subjects achieved a BMI of less than 27 kg/m², i.e. no longer overweight or obese according to the international BMI classification, for once-weekly subcutaneous administration of semaglutide 7.2 mg compared to 2.4 mg as shown in the Examples herein. In some embodiments effects referred to herein are for subjects having (i) obesity or (ii) overweight and at least one co-morbidity such as type 2 diabetes. In some embodiments effects referred to herein are in subjects having obesity. In some embodiments effects referred to herein are in subjects without diabetes, such as without type 1 diabetes and without type 2 diabetes. In some embodiments effects referred to herein are in subjects having obesity and not diabetes. In some embodiments once-weekly subcutaneous administration of semaglutide 7.2 mg provided significantly greater weight loss compared to 2.4 mg in subjects having obesity and not diabetes. In some embodiments a significantly greater proportion of subjects achieved a body weight reduction of at least 15%, at least 20% and at least 25% for once-weekly subcutaneous administration of semaglutide 7.2 mg compared to 2.4 mg in subjects having obesity and not diabetes. In some embodiments a significantly greater proportion of subjects achieved a BMI of less than 27 kg/m², i.e. no longer overweight or obese according to the international BMI classification, for once-weekly subcutaneous administration of semaglutide 7.2 mg compared to 2.4 mg in subjects having obesity and not diabetes. In some embodiments the present invention surprisingly provides a safe and tolerable medical therapy for weight management, including overweight and obesity, which provides an increased body weight reduction. In some embodiments the present invention surprisingly provides a safe and tolerable medical therapy for obesity which provides an increased body weight reduction.

Blood pressure reduction, both for systolic blood pressure and diastolic blood pressure, was surprisingly greater for once-weekly subcutaneous administration of semaglutide 7.2 mg compared to semaglutide 2.4 mg or placebo as shown in the Examples herein. Blood pressure reduction, both for systolic blood pressure and diastolic blood pressure, was surprisingly greater for once-weekly subcutaneous administration of semaglutide 7.2 mg compared to semaglutide 2.4 or placebo as shown in the Examples herein. In some embodiments the present invention surprisingly provides a safe and tolerable medical therapy for weight management, including overweight and obesity.

It was furthermore surprisingly found that a smaller than expected lean body volume compared to fat volume was lost with semaglutide, incl. semaglutide 7.2 mg, as shown in the Examples herein.

In some embodiments the invention is directed to methods for weight management of a human subject in need thereof comprising subcutaneous administration of about 7.2 mg semaglutide per week. In some embodiments the weight management comprising subcutaneous administration of about 7.2 mg semaglutide once weekly. In some embodiments the invention is directed to treatment of obesity or overweight comprising subcutaneous administration of about 7.2 mg semaglutide once weekly to a subject in need thereof. In some embodiments the invention is directed to treatment of obesity comprising subcutaneous administration of about 7.2 mg semaglutide once weekly to a subject in need thereof. In some embodiments the invention is directed to treatment of overweight comprising subcutaneous administration of about 7.2 mg semaglutide once weekly to a subject in need thereof. In some embodiments treatment of overweight and the subject further has at least one weight-related co-morbidity.

In some embodiments the invention is directed to methods for weight management of a human subject in need thereof comprising subcutaneous administration of about 7.2 mg semaglutide per week, wherein semaglutide provides a body weight reduction of at least about 15%, such as at least 18% or at least 20%. In some embodiments the invention is directed to methods for treatment of obesity or overweight, wherein for the treatment of overweight the subject may have at least one weight-related co-morbidity. In some embodiments the invention is directed to methods for treatment of obesity or overweight, wherein the subcutaneous administration further comprises one or more dosage escalation steps of administration of up to about 2.4 mg semaglutide per week, prior to administration of the about 7.2 mg semaglutide per week, and for the treatment of overweight the subject may have at least one weight-related co-morbidity. In some embodiments the invention is directed to methods for treatment of obesity or overweight, wherein the semaglutide is administered in the form of a pharmaceutical composition comprising semaglutide and the composition has a pH in the range of about 5.6 to about 9.0, and for the treatment of overweight the subject may have at least one weight-related co-morbidity. In some embodiments the invention is directed to methods for treatment of obesity or overweight, wherein the method does not comprise administration of cyclodextrin, and for the treatment of overweight the subject may have at least one weight-related co-morbidity.

In some embodiments the invention is directed to methods for weight management of a human subject in need thereof comprising subcutaneous administration of about 7.2 mg semaglutide once weekly, wherein the semaglutide provides a body weight reduction of at least about 15%, such as at least 18% or at least 20%. In some embodiments the invention is directed to methods for treatment of obesity or overweight, wherein for the treatment of overweight the subject may have at least one weight-related co-morbidity. In some embodiments the invention is directed to methods for treatment of obesity or overweight, wherein the subcutaneous administration further comprises one or more dosage escalation steps of administration of up to about 2.4 mg semaglutide once weekly, prior to administration of about 7.2 mg semaglutide once weekly, and for the treatment of overweight the subject may have at least one weight-related co-morbidity. In some embodiments the invention is directed to methods for treatment of obesity or overweight, wherein the semaglutide is administered in the form of a pharmaceutical composition comprising semaglutide and the composition has a pH in the range of about 5.6 to about 9.0, and for the treatment of overweight the subject may have at least one weight-related co-morbidity. In some embodiments the invention is directed to methods for treatment of obesity or overweight, wherein the method does not comprise administration of cyclodextrin, and for the treatment of overweight the subject may have at least one weight-related co-morbidity.

In some embodiments the invention is directed to methods for treating or reducing the risk of cardiovascular disease in a human subject in need thereof comprising subcutaneous administration of about 7.2 mg semaglutide per week. In some embodiments the invention is directed to methods for reducing the risk of major adverse cardiovascular event comprising CV death, non-fatal myocardial infarction and non-fatal stroke in a human subject in need thereof comprising subcutaneous administration of about 7.2 mg semaglutide per week. In some embodiments the invention is directed to methods for treating or reducing the risk of heart failure, such as heart failure with preserved ejection fraction, in a human subject in need thereof comprising subcutaneous administration of about 7.2 mg semaglutide per week. In some embodiments heart failure is heart failure with preserved ejection fraction. In some embodiments the invention is directed to methods for treating peripheral arterial disease in a human subject in need thereof comprising subcutaneous administration of about 7.2 mg semaglutide per week.

In some embodiments the invention is directed to methods for treating metabolic dysfunction-associated steatohepatitis (MASH) in a human subject in need thereof comprising subcutaneous administration of about 7.2 mg semaglutide per week. Metabolic dysfunction-associated steatohepatitis (MASH) is also known as non-alcoholic steatohepatitis (NASH).

In some embodiments the invention is directed to methods for treating type 2 diabetes in a human subject in need thereof comprising subcutaneous administration of about 7.2 mg semaglutide per week.

In some embodiments, the invention is directed to methods for treating Alzheimer's disease in a human subject in need thereof comprising subcutaneous administration of about 7.2 mg semaglutide per week. In some embodiments the Alzheimer's disease is early Alzheimer's disease.

In some embodiments the invention is directed to methods for treating chronic kidney disease in a human subject in need thereof comprising subcutaneous administration of about 7.2 mg semaglutide per week.

In some embodiments the term “treatment” as used herein refers to medical therapy by administration of a specified active ingredient, such as semaglutide, in an amount and frequency to provide a therapeutic effect which is preventative (“prevention”), palliative, symptomatic and/or curative. In some embodiments the treatment is symptomatic and/or curative. In some embodiments the treatment is prevention, including preventing a condition, such as obesity, from worsening.

The subject is human and may have obesity or overweight. In some embodiments the subject has obesity. In some embodiments the subject has overweight. In some embodiments the subject has overweight and at least one weight-related co-morbidity. In some embodiments the weight-related co-morbidity is selected from the group consisting of diabetes, cardiovascular disease, metabolic dysfunction-associated steatohepatitis, alcoholic liver disease, and obstructive sleep apnoea.

BMI (body mass index) is calculated as the weight of a subject in kilograms (kg) divided by the height of said subject in meters squared (m²). Unless otherwise mentioned, references herein to BMI, are based on the international classification as used in e.g. the US and Europe. In some embodiments the subject has a BMI of at least 30.

The term “obesity” may be defined herein as a subject with a BMI of at least 30 kg/m². In some embodiments, based on the Chinese classification, obesity is defined herein as a subject with a BMI of at least 28 kg/m². In some embodiments, based on the Japanese classification, obesity is defined herein as a subject with a BMI of at least 25 kg/m². In some embodiments, based on the Taiwanese classification, obesity is defined herein as a subject with a BMI of at least 27 kg/m².

The term “overweight” may be defined herein as a subject with a BMI of at least 27 kg/m². In some embodiments, based on the Chinese classification, overweight is defined herein as a subject with a BMI of at least 24 kg/m², such as at least 24 kg/m² to less than 28 kg/m². In some embodiments, based on the Taiwanese classification, overweight is defined herein as a subject with a BMI of at least 24 kg/m², such as at least 24 kg/m² to less than 27 kg/m².

In some embodiments the subject has a BMI of least 30 kg/m². The subject may have a BMI of least 35 kg/m². The subject may have a BMI of least 40 kg/m². The subject may have a BMI of 30-50 kg/m². The subject may have a BMI of 30-45 kg/m². The subject may have a BMI of 30-40 kg/m². The subject may have a BMI of 30-35 kg/m². The subject may have of at least 27 kg/m². The subject may have a BMI of 27-30 kg/m².

The subject may have a BMI of least 30 kg/m² and a body weight of at least about 100 kg. The subject may have a BMI of least 30 kg/m² and a body weight of at least about 120 kg. The subject may have a BMI of least 30 kg/m² and a body weight of at least about 140 kg.

The subject may be adult or paediatric. The subject may be adult. The subject may be paediatric. In some embodiments the subject is a paediatric (e.g. age 5 to less than 18), such as an adolescent (e.g. age 12 to less than 18). For paediatrics, obesity in may defined as subject with a BMI corresponding to at least 30 kg/m² for adults as defined in Table 4 of Cole et al., BMJ. 2000 May 6; 320(7244):1240 (doi: 10.1136/bmj.320.7244.1240). The subject may be a paediatric aged 12 years or older. The subject may be paediatric and with a BMI corresponding to of least 30 kg/m² for adults.

The term “lean body” as defined herein may be defined as the total body minus the total fat in the part of the body analysed, e.g. lean body volume may be total body volume minus the total fat volume. The term “total fat” as defined herein may be defined as sum of all fat in the part of the body analysed, e.g. from vertebrae T9 to the knees. The term “visceral fat” as defined herein may be defined as the fat located inside the abdominal cavity; thus, visceral fat includes fat surrounding organs in the abdominal cavity such as the liver, pancreas and kidney.

In some embodiments the subject has diabetes, such as type 2 diabetes. In some embodiments the subject has type 2 diabetes. In some embodiments the subject has at least one weight-related comorbid condition, such as dyslipidemia or hypertension. The subject may have overweight and at least one weight-related comorbid condition, such as dyslipidemia or hypertension. The subject may have a BMI of at least 27 kg/m² and at least one weight-related comorbid condition, such as dyslipidemia or hypertension. In some embodiments the subject is not classified as New York Heart Association (NYHA) class IV.

In some embodiments the weight management is the treatment of overweight or obesity in a human subject in need thereof, wherein the subject may have at least one weight-related co-morbidity. The term “weight-related co-morbidity” includes hyperglycaemia, type 2 diabetes, hypertension, dyslipidaemia, obstructive sleep apnoea, metabolic dysfunction-associated steatohepatitis, and/or cardiovascular disease. In some embodiments the weight management is the treatment of overweight in a human subject in need thereof, wherein the subject may have at least one weight-related co-morbidity. In some embodiments the weight management is the treatment of obesity in a human subject in need thereof, wherein the subject may have at least one weight-related co-morbidity. In some embodiments the weight management is adjunct to a reduced-calorie diet and increased physical activity. In some embodiments the weight management in a subject in need thereof is adjunct to a reduced-calorie diet and increased physical activity and the subject is obese. In some embodiments the weight management in a subject in need thereof is adjunct to a reduced-calorie diet and increased physical activity and the subject is overweight and has at least one weight-related co-morbidity. In some embodiments the weight management in a subject in need thereof is adjunct to a reduced-calorie diet and increased physical activity and the subject has a BMI of at least 30 kg/m², such as at least 28 kg/m² or at least 25 kg/m². In some embodiments the weight management in a subject in need thereof is adjunct to a reduced-calorie diet and increased physical activity and the subject has a BMI of at least 27 kg/m², such as at least 24 kg/m², and at least one weight-related co-morbidity.

In some embodiments the method of the invention provides a body weight reduction of the subject.

In some embodiments the body weight reduction is at least about 15%, such as at least 18% or at least 20%. In some embodiments the semaglutide provides a body weight reduction of at least about 15%, such as at least 18% or at least 20%. In some embodiments the body weight reduction is at least about 15%. In some embodiments the body weight reduction is at least 18%. In some embodiments the body weight reduction is at least 20%. In some embodiments the body weight reduction is at least 25%.

In some embodiments semaglutide provides a body weight reduction of at least about 15%, such as at least 18% or at least 20%. In some embodiments the about 7.2 mg semaglutide per week provides a body weight reduction of at least about 15%, such as at least 18% or at least 20%. In some embodiments the about 7.2 mg semaglutide per week administered for at least 52 weeks, such as at least about 72 weeks including dosage escalation, provides a body weight reduction of at least about 15%, such as at least 18% or at least 20%. In some embodiments the about 7.2 mg semaglutide per week administered for at least 52 weeks provides a body weight reduction of at least about 15%, such as at least 18% or at least 20%. In some embodiments the about 7.2 mg semaglutide per week administered for at least about 72 weeks including dosage escalation, provides a body weight reduction of at least about 15%, such as at least 18% or at least 20%. In some embodiments the about 7.2 mg semaglutide per week including administered for at least 52 weeks provides a body weight reduction of at least about 15%. In some embodiments the about 7.2 mg semaglutide per week including administered for at least 52 weeks provides a body weight reduction of at least about 20%. In some embodiments the about 7.2 mg semaglutide per week including administered for at least 52 weeks provides a body weight reduction of at least about 25%. In some embodiments the body weight reduction is at least about 15% after at least about 52 weeks of administration of the about 7.2 mg semaglutide. In some embodiments the body weight reduction is about 15% after about 52 weeks of administration of the about 7.2 mg semaglutide. In some embodiments the body weight reduction is at least 15% after about 52 weeks of administration of the 7.2 mg semaglutide. In some embodiments the body weight reduction is at least 18% after about 52 weeks of administration of the 7.2 mg semaglutide. In some embodiments the body weight reduction is at least 20% after at least about 52 weeks of administration of the about 7.2 mg semaglutide. In some embodiments the body weight reduction is at least 25% after at least about 52 weeks of administration of the about 7.2 mg semaglutide. In some embodiments the body weight reduction is by up to 75%. In some embodiments the body weight reduction is by up to 75% after at least about 52 weeks of administration of the about 7.2 mg semaglutide.

In some embodiments a greater proportion of subjects obtain a body weight reduction of at least 20% is greater by administration of the 7.2 mg semaglutide once weekly compared to 2.4 mg semaglutide once weekly. In some embodiments at least 45%, such as about 50%, of subjects obtain a body weight reduction of at least 20% with the 7.2 mg semaglutide. In some embodiments at least 45%, such as about 50%, of subjects obtain a body weight reduction of at least 20% with the 7.2 mg semaglutide once weekly. In some embodiments the a greater proportion of subjects obtain a body weight reduction of at least 25% is greater by administration of the about 7.2 mg semaglutide once weekly compared to 2.4 mg semaglutide once weekly. In some embodiments at least 25%, such as at least 30%, such as about 33%, of subjects obtain a body weight reduction of at least 25% with the about 7.2 mg semaglutide. In some embodiments at least 30%, such as about 33%, of subjects obtain a body weight reduction of at least 25% with the about 7.2 mg semaglutide once weekly. In some embodiments a greater proportion of subjects obtain a BMI of less than 27 kg/m². In some embodiments a greater proportion of subjects obtain a BMI of less than 27 kg/m² with the administration of about 7.2 mg semaglutide per week compared to about 2.4 mg semaglutide per week. In some embodiments a greater proportion of subjects obtain a BMI of less than 27 kg/m² with the administration of 7.2 mg semaglutide once weekly compared to 2.4 mg semaglutide once weekly. In some embodiments at least about 25%, such as about 27%, of subjects obtain a BMI of less than 27 kg/m² with the 7.2 mg semaglutide. In some embodiments a greater proportion of subjects obtain a BMI of less than 27 kg/m² with the administration of about 7.2 mg semaglutide per week compared to about 2.4 mg semaglutide per week. In some embodiments a greater proportion of subjects obtain a BMI of less than 27 kg/m² with the administration of 7.2 mg semaglutide once weekly compared to 2.4 mg semaglutide once weekly.

In some embodiments the method provides a reduction of lean body volume compared to total fat volume of less than 20%, such as less than 18%, such as less than 17%, or less than 16%. In some embodiments the method provides a reduction of lean body volume compared to total fat volume of no more than 16.5%. In some embodiments the method provides a reduction of lean body volume compared to total fat volume of no more than 16%. In some embodiments the method provides a reduction of lean body volume compared to total fat volume of no more than 15.5%. In some embodiments the method provides a reduction of lean body volume compared to total fat volume of no more than 15%. In some embodiments the method provides a reduction of lean body volume compared to total fat volume of about 10% to about 20%.

In some embodiments a greater proportion of subjects obtain a waist-to-height ratio of less than 0.50 is greater by administration of the 7.2 mg semaglutide once weekly compared to 2.4 mg semaglutide once weekly. In some embodiments the method provides a waist-to-height ratio of less than 0.50 in at least about 10% of subjects. In some embodiments at least 10%, such as at least about 12% or at least about 14%, of subjects obtain a waist-to-height ratio of less than 0.50 with the 7.2 mg semaglutide once weekly. Waist-to-height ratio may be calculated as a subject's waist circumference divided by its height.

In some embodiments the method is tolerable for the subject. In some embodiments less than about 15% of subjects discontinue administration of the about 7.2 mg semaglutide. In some embodiments about 8% to about 15% of subjects discontinue administration of the about 7.2 mg semaglutide. In some embodiments less than about 15% of subjects discontinue administration of the about 7.2 mg semaglutide within at least about 52 weeks. In some embodiments less than 15% of subjects discontinue administration of the about 7.2 mg semaglutide within at least about 52 weeks after an initial dosage escalation, wherein the dosage escalation is as defined herein. In some embodiments less than 15% of subjects discontinue administration of the about 7.2 mg semaglutide within at least about 52 weeks after an initial dosage escalation of up to about 2.4 mg semaglutide once weekly. In some embodiments less than 15% of subjects discontinue administration of the about 7.2 mg semaglutide within up to about 72 weeks from the initial semaglutide dosage escalation step, such as an initial semaglutide dosage escalation step of 0.25 mg semaglutide once weekly. In some embodiments less than 15% of subjects discontinue administration of the about 7.2 mg semaglutide within up to about 72 weeks from the initial semaglutide dosage escalation step, such as an initial semaglutide dosage escalation step of 0.25 mg semaglutide once weekly, and the method comprising dosage escalation of up to about 2.4 mg semaglutide once weekly. In some embodiments less than 12% of subjects discontinue administration of the about 7.2 mg semaglutide. In some embodiments less than 12% of subjects discontinue administration of the about 7.2 mg semaglutide within at least about 52 weeks. In some embodiments less than 12% of subjects discontinue administration of the about 7.2 mg semaglutide within at least about 52 weeks after an initial dosage escalation, wherein the dosage escalation is as defined herein. In some embodiments less than 12% of subjects discontinue administration of the about 7.2 mg semaglutide within at least about 52 weeks after an initial dosage escalation of up to about 2.4 mg semaglutide once weekly. In some embodiments less than 12% of subjects discontinue administration of the about 7.2 mg semaglutide within up to about 72 weeks from the initial semaglutide dosage escalation step, such as an initial semaglutide dosage escalation step of 0.25 mg semaglutide once weekly. In some embodiments less than 12% of subjects discontinue administration of the about 7.2 mg semaglutide within up to about 72 weeks from the initial semaglutide dosage escalation step, such as an initial semaglutide dosage escalation step of 0.25 mg semaglutide once weekly, and the method comprising dosage escalation of up to about 2.4 mg semaglutide once weekly. In some embodiments “discontinue” is a permanent discontinuation, such as a discontinuation for at least 4 weeks or at least 8 weeks. In some embodiments less than about 15% of subjects discontinue administration of the about 7.2 mg semaglutide and the semaglutide provides a body weight reduction of at least about 15%. In some embodiments about 65% to about 75% of subjects have at least one gastrointestinal adverse event. In some embodiments about 65% to about 75% of subjects have at least one gastrointestinal adverse event during administration of the about 7.2 mg semaglutide within about 52 weeks. In some embodiments about 71% of subjects have at least one gastrointestinal adverse event during administration of the about 7.2 mg semaglutide within about 52 weeks. In some embodiments about 71% of subjects have at least one gastrointestinal adverse event during administration of the about 7.2 mg semaglutide within about 72 weeks including dosage escalation. In some embodiments about 65% to about 75% of subjects have at least one gastrointestinal adverse event. In some embodiments about 35% to about 50% of subjects have at least one gastrointestinal adverse event. In some embodiments about 40% to about 48% of subjects have at least one gastrointestinal adverse event. In some embodiments about 44% of subjects have at least one gastrointestinal adverse event.

In some embodiments the waist circumference of the subject is reduced.

In some embodiments the blood pressure of the subject is reduced. In some embodiments the diastolic blood pressure and/or systolic blood pressure of the subject is reduced. In some embodiments the diastolic blood pressure and systolic blood pressure of the subject is reduced.

In some embodiments the invention is directed to semaglutide for use in weight management, such as treatment of obesity or overweight, comprising subcutaneous administration of about 7.2 mg semaglutide per week to a human subject.

In some embodiments the invention is directed to use of semaglutide in the manufacture of a medicament for use in weight management, such as treatment of obesity or overweight, comprising subcutaneous administration of about 7.2 mg semaglutide per week to a human subject.

In some embodiments the invention is directed to a pharmaceutical composition comprising semaglutide formulated for subcutaneous administration of about 7.2 mg semaglutide per week. In some embodiments the invention is directed to a pharmaceutical composition comprising semaglutide formulated for subcutaneous administration of about 7.2 mg semaglutide per week a human subject with overweight or obesity.

In some embodiments the invention is directed to a kit comprising semaglutide or a pharmaceutical acceptable salt thereof. In some embodiments the invention is directed to a kit comprising semaglutide and instructions for use wherein the semaglutide is for subcutaneous administration of about 7.2 semaglutide per week. In some embodiments the invention is directed to a kit comprising semaglutide and instructions for use wherein the semaglutide is for subcutaneous administration of about 7.2 semaglutide per week, wherein semaglutide is contained in a vial. In some embodiments the invention is directed to a kit comprising semaglutide and instructions for use wherein the semaglutide is for subcutaneous administration of about 7.2 semaglutide per week, wherein semaglutide is contained in an injection device.

### Semaglutide

The method of the invention comprises the GLP-1 receptor agonist semaglutide. Semaglutide is N-epsilon26-[2-(2-{2-[2-(2-{2-[(S)-4-Carboxy-4-(17-carboxyheptadecanoyl-amino)butyrylamino]ethoxy}ethoxy) acetylamino]ethoxy}ethoxy) acetyl] [Aib8,Arg34] GLP-1-(7-37) and may be prepared as described in Example 4 of WO2006/097537, the contents of which are incorporated by reference in their entirety. Semaglutide may be in the form of a salt, such as a pharmaceutically acceptable salt. In some embodiments the term “GLP-1 receptor agonist” as used herein refers to any compound, including peptides and non-peptide compounds, which binds to the human GLP-1 receptor with a potency (EC₅₀) of below 100 nM as determined by methods known in the art, see for example WO98/08871, the contents of which are incorporated by reference in their entirety. In some embodiments methods for identifying GLP-1 receptor agonists are described in WO93/19175, the contents of which are incorporated by reference in their entirety.

In some embodiments semaglutide is the sole active ingredient administered in the methods of the invention. In some embodiments the method of the invention comprises administering one or more additional active ingredients which are not GLP-1 receptor agonists to the subject.

### Administration Regimen

Semaglutide is administered by subcutaneous administration. In some embodiments semaglutide is administered via subcutaneous injection. In some embodiments semaglutide is contained in a vial. In some embodiments semaglutide is contained in an injection device. In some embodiments semaglutide is administered via an injection device. In some embodiments semaglutide is administered using a pen-injector, such as a 3 ml disposable pen-injector.

In some embodiments semaglutide is administered in an amount of about 7.2 mg semaglutide per week. In some embodiments semaglutide is administered in an amount of about 7.2 mg semaglutide once weekly. In some embodiments semaglutide is administered in an amount of 7.2 mg semaglutide per week.

In some embodiments semaglutide is administered once weekly. In some embodiments semaglutide is administered in an amount of about 7.2 mg semaglutide once weekly. In some embodiments semaglutide is administered in an amount of 7.2 mg semaglutide once weekly.

In some embodiments semaglutide is administered at a fixed dose, rather than a weight-based dose, with or without dosage escalation. This fixed dose (e.g., about 7.2 mg semaglutide) applies to different subject groups (varying in weight, gender, disease severity, BMI, etc.), without individual dose adjustment. This simplifies the treatment regimen and enhances patient compliance.

In some embodiments semaglutide is administered for at least 24 weeks. Semaglutide may be administered for at least 32 weeks. Semaglutide may be administered for at least 40 weeks. Semaglutide may be administered for at least about 52 weeks. Semaglutide may be administered for at least about 72 weeks. Semaglutide may be administered for about 72 weeks. Semaglutide may be administered for at least about 72 weeks from the initial semaglutide dosage escalation step. Semaglutide may be administered for up to about 72 weeks from the initial semaglutide dosage escalation step. Semaglutide may be administered for as at least about 72 weeks including dosage escalation. Semaglutide may be administered for as about 72 weeks including dosage escalation. Semaglutide may be administered for at least about 52 weeks of the of about 7.2 mg semaglutide per week.

In some embodiments semaglutide is be administered once daily or less frequently, such as once daily or once weekly. In some embodiments the GLP-1 receptor agonist is administered at any time in the day. In some embodiments the method of the invention is continued for at least about 4 weeks, such as at least about 5 weeks.

In some embodiments the method according to the invention is for chronic management. In some embodiments the term “chronic management” as used herein refers to continuing administering semaglutide according to the method of the invention for an extended period of time, such as at least 1 year or longer. In some embodiments, the term “chronic management” as used herein refers to continuing administering semaglutide according to the method of the invention for at least 5 years, such as at least 10 years. In some embodiments, the term “chronic management” as used herein refers to continuing administering semaglutide according to the method of the invention for at least 15 years or at least 20 years. In some embodiments, the term “chronic management” as used herein refers to continuing administering semaglutide according to the method of the invention for the remaining lifetime of a subject. In some embodiments the method according to the invention is continued for at least about 48 weeks. In some embodiments chronic management comprises administration of semaglutide in an amount and frequency, e.g. as specified herein, sufficient for the treatment of obesity.

In some embodiments semaglutide is the sole GLP-1 receptor agonist administered in the methods of the invention. In some embodiments semaglutide is the only active ingredient administered in the methods of the invention. In some embodiments the method of the invention comprises administering one or more additional active ingredients which are not GLP-1 receptor agonists to the subject.

### Dosage Escalation

Administration of semaglutide may be initiated via dosage escalation, i.e. beginning with an amount lower than the treatment dosage and gradually increasing towards the treatment dosage over time. Dosage escalation may help avoid one or more unwanted side effects. As used herein, the term “treatment dosage” refers to the dosage (i.e. amount and administration frequency) of semaglutide. In some embodiments the treatment dosage results in therapeutic effect in the medical indication referred to. In some embodiments the method of the invention comprises an initial step of dosage escalation, wherein the subject is administered (i) a dosage of semaglutide in the range of from about one tenth to half of the treatment dosage, and then (ii) every 2-6 weeks, such as about every 2 weeks, about every 4 weeks, or about every 5 weeks, dosage is increased by 1.5-2.5 times, such as about 2 times, until at treatment dosage. In some embodiments the method of the invention comprises an initial step of dosage escalation, wherein the subject is administered (i) a dosage of semaglutide in the range of 0.2-0.5 mg semaglutide per week, such as about 0.35 mg semaglutide per week, and then (ii) every 2-6 weeks, such as about every 4 weeks, dosage is increased by 1.5-2.5 times, such as about 2 times, until at treatment dosage. In some embodiments, and unless otherwise specified, amounts and/or administration frequency for semaglutide mentioned herein refer to the treatment dosage. In some embodiments the method of the invention comprises a dosage escalation, such as a further dosage escalation, from about 2.4 mg semaglutide per week to about 7.2 mg semaglutide per week. In some embodiments the treatment dosage for subcutaneous administration is about 7.2 mg semaglutide per week. In some embodiments the treatment dosage is about 2.4 mg semaglutide per week.

In some embodiments the subcutaneous administration further comprises one or more dosage escalation steps of up to about 2.4 mg semaglutide per week followed by a dosage of the about 7.2 mg semaglutide per week. In some embodiments the subcutaneous administration further comprises one or more dosage escalation steps of about 0.25 mg to about 2.4 mg semaglutide per week followed by a dosage of the about 7.2 mg semaglutide per week. In some embodiments the subcutaneous administration comprises a dosage escalation step of about 2.4 mg semaglutide per week followed by a dosage of the about 7.2 mg semaglutide per week. In some embodiments the method further comprises one or more dosage escalation steps of administration of up to about 2.4 mg semaglutide per week, prior to administration of the about 7.2 mg semaglutide per week. In some embodiments the administration comprises a dosage escalation step of administration of about 2.4 mg semaglutide per week, prior to administration of the about 7.2 mg semaglutide per week. In some embodiments the subcutaneous administration comprises dosage escalation steps for up to about 20 weeks, such as for about 20 weeks. In some embodiments the subcutaneous administration comprises dosage escalation steps for up to about 20 weeks at no more than about 2.4 mg semaglutide per week followed by a dosage of the about 7.2 mg semaglutide per week. In some embodiments the subcutaneous administration comprises dosage escalation steps every about four weeks from about 0.25 mg semaglutide per week, to about 0.5 mg semaglutide per week, to about 1.0 mg semaglutide per week, to about 1.7 mg semaglutide per week, to about 2.4 mg semaglutide per week followed by a dosage of the about 7.2 mg semaglutide per week. In some embodiments the method comprises a dosage escalation step at about 2.4 mg semaglutide per week one week prior to the first administration of about 7.2 mg semaglutide per week. In some embodiments the method comprises a dosage escalation step at 2.4 mg semaglutide once weekly one week prior to the first administration of 7.2 mg semaglutide once weekly. In some embodiments the method comprises a dosage escalation steps from weekly semaglutide administration in an amount of about (i) 0.25 mg to (ii) 0.5 mg to (iii) 1.0 mg to (iv) 1.7 mg to (v) 2.4 mg prior to administration of the about 7.2 mg semaglutide per week. In some embodiments the method comprises a dosage escalation steps from once weekly semaglutide administration in an amount of about (i) 0.25 mg to (ii) 0.5 mg to (iii) 1.0 mg to (iv) 1.7 mg to (v) 2.4 mg prior to administration of the about 7.2 mg semaglutide once weekly. In some embodiments the method comprises a dosage escalation steps about every four weeks from once weekly semaglutide administration in an amount of about (i) 0.25 mg to (ii) 0.5 mg to (iii) 1.0 mg to (iv) 1.7 mg to (v) 2.4 mg prior to administration of the about 7.2 mg semaglutide once weekly. In some embodiments the method comprises a dosage escalation steps every four weeks from once weekly semaglutide administration in an amount of (i) 0.25 mg to (ii) 0.5 mg to (iii) 1.0 mg to (iv) 1.7 mg to (v) 2.4 mg prior to administration of the 7.2 mg semaglutide once weekly.

In some embodiments the invention provides a pharmaceutical combination, comprising 1) a first dose comprising about 0.25 mg semaglutide; and/or 2) a second dose comprising about 0.5 mg semaglutide; 3) and/or a third dose comprising about 1.0 mg semaglutide; 4) and/or a fourth dose comprising about 1.7 mg semaglutide; 5) and/or a fifth dose comprising about 2.4 mg semaglutide; and 6) a sixth dose comprising about 7.2 mg semaglutide.

In some embodiments, each dose escalates every four weeks from once weekly semaglutide administration in an amount of (i) 0.25 mg to (ii) 0.5 mg to (iii) 1.0 mg to (iv) 1.7 mg to (v) 2.4 mg prior to administration of 7.2 mg semaglutide once weekly. In some embodiments, each dose comprises 1 to 4 single weekly doses, preferably, each dose comprises 1 single weekly dose, or preferably, each dose comprises 4 single weekly doses.

In some embodiments, one or more doses of semaglutide is administered weekly to obtain 7.2 mg semaglutide weekly. In some embodiments, 7.2 mg semaglutide is administered once weekly by administering one or more doses of semaglutide that combined result in 7.2 mg semaglutide weekly. In some embodiments, one or more doses of semaglutide can be any combination that results in 7.2 mg semaglutide, e.g., but not limited to, administration of 2.4 mg semaglutide three times. In some embodiments, 2.4 mg semaglutide is administered three consecutive times, e.g., administered just following each other, e.g., within a shout time period, such as 1-100 minutes, such as 1-5 minutes.

### Further Active Ingredient

In some embodiments the method of the invention comprises administration of one or more further active ingredients.

In some embodiments the method of the invention further comprises administration of an amylin receptor agonist. An amylin receptor agonist may bind to and activate the calcitonin receptor (CTR) and/or the amylin receptors (AMYRs). The AMYRs consist of heterodimers of two components: the calcitonin receptor (CTR) and one of three receptor activity-modifying proteins (RAMP1-3) resulting in three possible complexes, AMYR1-3. An “amylin receptor agonist” may be defined herein as a chemical entity which is capable of binding to an amylin receptor and is capable of activating; in other words, it is “potent” on the amylin receptor. In the context of the current invention, the amylin receptor agonist is capable of binding to and activating at least the amylin receptor 3 (AMYR3). The amylin receptor agonist may also be capable of activating the calcitonin receptor, the amylin receptor 1 (AMYR1) and/or the amylin receptor 2 (AMYR2). The in vitro potency of the amylin receptor agonist on amylin receptor 3 may be determined as described in WO2022/129526, Assay 2. The potency of the compound may be described by means of its EC₅₀ value, wherein EC₅₀ represents the concentration of compound upon which 50% of its maximal effect is observed. The lower the EC₅₀ value, the more potent the compound. When tested as described as described in WO2022/129526, Assay 2, the amylin receptor agonist as disclosed herein may have an EC₅₀ value of less than 300 pM, such as less than 200 pM, such as less than 150 pM, preferably less than 100 pM, such as less than 75 pM, preferably less than 50 pM, such as less than 40 pM, such as less than 30 pM, such as less than 20 pM, such as less than 10 pM.

The amylin receptor agonist may be cagrilintide or a biologically active metabolite or degradation product of cagrilintide. In some embodiments the method of the invention further comprises administration of cagrilintide. Cagrilintide, also known as AM833, is the compound of Example 53 in WO2012/168432: N-alpha-[(S)-4-Carboxy-4-(19-carboxynonadecanoylamino)butyryl]-[Glu14,Arg17, Pro37]-pramlintide. Cagrilintide may be prepared as described in WO2012/168432, pages 153-155. Cagrilintide may be in the form of a salt, such as a pharmaceutically acceptable salt. A biologically active metabolite or degradation product of cagrilintide may have an aspartate (Asp) in position 21 or 22. A biologically active metabolite or degradation product of cagrilintide may have an iso-aspartate (iso-Asp) in position 21 or 22. When the potency of cagrilintide was tested using the procedure described in WO2022129526, Assay 2, cagrilintide had an EC₅₀ value of about 11 pM (WO2022/129526, Tables 4b and 4c).

In some embodiments the method further comprises administration of cagrilintide. In some embodiments cagrilintide is administered simultaneously with or sequentially to the semaglutide. In some embodiments cagrilintide is administered via subcutaneous administration. In some embodiments semaglutide and cagrilintide are administered via subcutaneous administration. In some embodiments cagrilintide is administered via subcutaneous injection. In some embodiments semaglutide and cagrilintide are administered via subcutaneous injection.

In some embodiments cagrilintide is administered in amount of about 0.25-16 mg per week. In some embodiments cagrilintide is administered in amount of about 0.25-9 mg per week. In some embodiments cagrilintide is administered in amount of about 0.25-4.5 mg per week. In some embodiments cagrilintide is administered in amount of about 0.25-3.6 mg per week. In some embodiments cagrilintide is administered in amount of about 0.25-2.4 mg per week. In some embodiments cagrilintide is administered in amount of about 0.25 mg per week. In some embodiments cagrilintide is administered in amount of 0.5 mg per week. In some embodiments cagrilintide is administered in amount of 1.0 mg per week. In some embodiments cagrilintide is administered in amount of 1.7 mg per week. In some embodiments cagrilintide is administered in amount of 2.4 mg per week. In some embodiments cagrilintide is administered in amount of 3.6 mg per week. In some embodiments cagrilintide is administered once weekly. In some embodiments cagrilintide is administered in amount of about 0.25-16 mg once weekly. In some embodiments cagrilintide is administered in amount of about 0.25 mg once weekly. In some embodiments cagrilintide is administered in amount of 0.5 mg once weekly. In some embodiments cagrilintide is administered in amount of 1.0 mg once weekly. In some embodiments cagrilintide is administered in amount of 1.7 mg once weekly. In some embodiments cagrilintide is administered in amount of 2.4 mg once weekly. In some embodiments cagrilintide is administered in amount of 3.6 mg once weekly.

In some embodiments cagrilintide is administered in the form of a pharmaceutical composition comprising cagrilintide and one or more excipients. In some embodiments cagrilintide is administered in the form of a pharmaceutical composition comprising cagrilintide, semaglutide and one or more excipients. In some embodiments semaglutide is administered in the form of a pharmaceutical composition consisting of cagrilintide, semaglutide and one or more excipients. In some embodiments pharmaceutical composition comprising cagrilintide is a solution. In some embodiments pharmaceutical composition comprising cagrilintide is an aqueous solution. In some embodiments pharmaceutical composition comprising cagrilintide and semaglutide has a pH in the range of 5.6-6.0. In some embodiments the pharmaceutical composition comprising cagrilintide and semaglutide has a pH in the range of 5.8-6.2. In some embodiments the pharmaceutical composition comprising cagrilintide has a pH of about 3.5-4.5, such as pH 4.0. In some embodiments the pharmaceutical composition comprising cagrilintide comprises a buffer. In some embodiments the pharmaceutical composition comprising cagrilintide comprises a glutamic acid, glutamate, lactic acid, lactate, acetic acid, or acetate buffer. In some embodiments pharmaceutical composition comprising cagrilintide comprises a glutamic acid or glutamate buffer. In some embodiments the pharmaceutical composition comprising cagrilintide comprises a lactic acid or lactate buffer. In some embodiments the pharmaceutical composition comprising cagrilintide comprises an acetic acid or acetate buffer.

In some embodiments administration of cagrilintide further comprises one or more dosage escalation steps, wherein the dosage escalation steps may be as defined herein for semaglutide. During dosage escalation, the ratio of cagrilintide to semaglutide may be about 1:1. Cagrilintide and semaglutide may be administered at an initial dosage escalation step of 0.25 mg per week, followed by further dosage escalation steps of 0.5 mg, 1.0 mg, 1.7 mg and 2.4 mg per week. Cagrilintide and semaglutide may be administered once weekly an initial dosage escalation step of 0.25 mg, followed by further dosage escalation steps of 0.5 mg, 1.0 mg, 1.7 mg and 2.4 mg.

After dosage escalation, the ratio of amylin receptor agonist to GLP-1 receptor agonist may be between 1:1 and 1:7. After dosage escalation, the ratio of cagrilintide to semaglutide may be between 1:1 and 1:7. In some embodiments, semaglutide is administered at a maintenance dosage of about 7.2 mg per week by subcutaneous administration and cagrilintide is administered at a maintenance dosage of about 2.4 mg per week by subcutaneous administration.

The dosage of cagrilintide may be about 0.25 mg and the dosage of semaglutide may be about 0.25 mg. The dosage of cagrilintide may be about 0.5 mg and the dosage of semaglutide may be about 0.5 mg. The dosage of cagrilintide may be about 1.0 mg and the dosage of semaglutide may be about 1.0 mg. The dosage of cagrilintide may be about 1.7 mg and the dosage of semaglutide may be about 1.7 mg. The dosage of cagrilintide may be about 2.4 mg and the dosage of semaglutide may be about 2.4 mg. The dosage of cagrilintide may be about 2.4 mg and the dosage of semaglutide may be about 6.0 mg.

The maintenance dosage of cagrilintide may be about 2.4 mg and the maintenance dosage of semaglutide may be about 7.2 mg. The maintenance dosage of cagrilintide may be about 2.4 mg and the maintenance dosage of semaglutide may be about 6.0 mg. The maintenance dosage of cagrilintide may be about 2.4 mg and the maintenance dosage of semaglutide may be about 6.9 mg. The maintenance dosage of cagrilintide may be about 2.4 mg and the maintenance dosage of semaglutide may be about 8.0 mg.

In some embodiments the invention is directed to methods for weight management of a human subject in need thereof comprising subcutaneous administration of about 7.2 mg semaglutide per week, wherein the semaglutide provides a body weight reduction of at least about 15%, such as at least 18% or at least 20%, and the method further comprises subcutaneous administration of about 0.25-16 mg cagrilintide per week. In some embodiments the invention is directed to methods for treatment of obesity or overweight, wherein for the treatment of overweight the subject may have at least one weight-related co-morbidity, and the method further comprises subcutaneous administration of about 0.25-16 mg cagrilintide per week. In some embodiments the invention is directed to methods for treatment of obesity or overweight, wherein the subcutaneous administration further comprises one or more dosage escalation steps of administration of up to about 2.4 mg semaglutide per week, prior to administration of the about 7.2 mg semaglutide per week, and for the treatment of overweight the subject may have at least one weight-related co-morbidity, and the method further comprises subcutaneous administration of about 0.25-16 mg cagrilintide per week. In some embodiments the invention is directed to methods for treatment of obesity or overweight, wherein the semaglutide is administered in the form of a pharmaceutical composition comprising semaglutide and the composition has a pH in the range of about 5.6 to about 9.0, and for the treatment of overweight the subject may have at least one weight-related co-morbidity, and the method further comprises subcutaneous administration of about 0.25-16 mg cagrilintide per week. In some embodiments the invention is directed to methods for treatment of obesity or overweight, wherein the method does not comprise administration of cyclodextrin, and for the treatment of overweight the subject may have at least one weight-related co-morbidity, and the method further comprises subcutaneous administration of about 0.25-16 mg cagrilintide per week.

In some embodiments the invention is directed to methods for weight management of a human subject in need thereof comprising subcutaneous administration of about 7.2 mg semaglutide once weekly, wherein the semaglutide provides a body weight reduction of at least about 15%, such as at least 18% or at least 20%, and the method further comprises subcutaneous administration of about 0.25-16 mg cagrilintide once weekly. In some embodiments the invention is directed to methods for treatment of obesity or overweight, wherein for the treatment of overweight the subject may have at least one weight-related co-morbidity, and the method further comprises subcutaneous administration of about 0.25-16 mg cagrilintide once weekly. In some embodiments the invention is directed to methods for treatment of obesity or overweight, wherein the subcutaneous administration further comprises one or more dosage escalation steps of administration of up to about 2.4 mg semaglutide per week, prior to administration of the about 7.2 mg semaglutide per week, and for the treatment of overweight the subject may have at least one weight-related co-morbidity, and the method further comprises subcutaneous administration of about 0.25-16 mg cagrilintide once weekly. In some embodiments the invention is directed to methods for treatment of obesity or overweight, wherein the semaglutide is administered in the form of a pharmaceutical composition comprising semaglutide and the composition has a pH in the range of about 5.6 to about 9.0, and for the treatment of overweight the subject may have at least one weight-related co-morbidity, and the method further comprises subcutaneous administration of about 0.25-16 mg cagrilintide once weekly. In some embodiments the invention is directed to methods for treatment of obesity or overweight, wherein the method does not comprise administration of cyclodextrin, and for the treatment of overweight the subject may have at least one weight-related co-morbidity, and the method further comprises subcutaneous administration of about 0.25-16 mg cagrilintide once weekly.

In some embodiments the method of the invention comprises administering semaglutide and one or more additional active ingredients which are not GLP-1 receptor agonists or amylin receptor agonists, such as cagrilintide, to the subject.

### Pharmaceutical Composition

In the method of the invention semaglutide may be administered in the form of a pharmaceutical composition, also referred to as a composition herein. In some embodiments semaglutide is the sole active ingredient in the pharmaceutical composition. In some embodiments the pharmaceutical composition comprises semaglutide and one or more excipients. In some embodiments the pharmaceutical composition consists essentially of semaglutide and one or more excipients. In some embodiments the pharmaceutical composition consists of semaglutide and one or more excipients. In some embodiments the method herein consists of administering a pharmaceutical composition consisting of semaglutide and one or more excipients.

Semaglutide may be administered in the form of the pharmaceutical composition described in WO2019/038412. Semaglutide may be administered in the form of the pharmaceutical composition described in WO2021/144477.

The pharmaceutical composition may comprise semaglutide in a concentration from 0.1 mg/ml to 100 mg/ml. In some embodiments the pharmaceutical composition comprises 0.01-50 mg/ml, or 0.01-20 mg/ml, or 0.01-10 mg/ml semaglutide. In some embodiments the pharmaceutical composition comprises 0.1-20 mg/ml semaglutide. The concentration of semaglutide in the pharmaceutical formulation may be from about 0.25 mg/ml to about 22 mg/ml.

In some embodiments the pharmaceutical composition comprises a unit dosage form of semaglutide. One unit dosage form may contain a single weekly dose. In some embodiments, a single weekly dose is up to 7.2 mg semaglutide, such as about 6.0 mg to about 7.2 mg semaglutide, such as about 7.2 mg semaglutide. In some embodiments, the pharmaceutical composition comprises one to four unit dosage forms of semaglutide. In some embodiments, a pharmaceutical composition comprises about 7.2 to 28.8 mg semaglutide, preferably, a pharmaceutical composition comprises about 7.2 mg or about 28.8 mg semaglutide, more preferably, a pharmaceutical composition comprises about 7.2 mg semaglutide. In some embodiments, a pharmaceutical composition is contained in an injection device or a vial.

In some embodiments, at dosage escalation steps, one unit dosage form of semaglutide comprises (i) 0.25 mg, (ii) 0.5 mg, (iii) 1.0 mg, (iv) 1.7 mg or (v) 2.4 mg semaglutide. In some embodiments, a pharmaceutical composition comprises one to four unit dosage forms of semaglutide. In some embodiments, a pharmaceutical composition is contained in an injection device or a vial.

In some embodiments, a pharmaceutical composition comprising 7.2 mg semaglutide may be replaced with three pharmaceutical compositions, each comprising unit dosage form of 2.4 mg semaglutide. Thus, in some embodiments, a pharmaceutical composition intended to comprise 7.2 mg semaglutide may alternatively use three pharmaceutical compositions, each comprising unit dosage form of 2.4 mg semaglutide. In this way, the fifth dose of 2.4 mg semaglutide can also be used to the sixth dose of 7.2 mg semaglutide by administration of three unit dosage forms.

The pharmaceutical compositions described herein may further comprise one or more pharmaceutically acceptable excipients, for example selected from the group consisting of buffer system, preservative, tonicity agent, chelating agent, stabilizer and surfactant. In some embodiments the pharmaceutical composition comprises one or more pharmaceutically acceptable excipients, such as one or more selected from the group consisting of a buffer, an isotonic agent, and a preservative. The formulation of pharmaceutically active ingredients with various excipients is known in the art, see e.g.

Remington: The Science and Practice of Pharmacy (e.g. 19th edition (1995), and any later editions). The term “excipient” broadly refers to any component other than the active therapeutic ingredient(s), e.g. semaglutide. The excipient may be an inert substance, an inactive substance, and/or a not medicinally active substance (also referred to herein as an active ingredient).

In some embodiments semaglutide is administered in the form of a pharmaceutical composition consisting of semaglutide and one or more excipients. In some embodiments semaglutide is administered in the form of a pharmaceutical composition consisting of semaglutide and one or more excipients and the composition has a pH in the range of about 5.6 to about 10.0.

In some embodiments the pharmaceutical composition has a pH in the range of about 6.5 to about 10.0, such as about 7.0 to about 9.5 or about 7.2 to about 9.0. In some embodiments the pharmaceutical composition has a pH in the range of about 7.0 to about 9.5 or about 7.2 to about 9.5. In some embodiments the pharmaceutical composition has a pH in the range of about 6.8 to about 9.0, such as about 7.0 to about 8.5 or about 7.0 to about 8.0. In some embodiments the pharmaceutical composition has a pH in the range of 7.0-8.5, such as about 6.8 to about 8.2, such as about 6.8 to about 7.8, or about 7.0 to about 7.6. The pharmaceutical composition may have a pH in the range of about 6.0 to about 9.0. The pharmaceutical composition may have a pH in the range of about 6.8 to about 8.0. The pharmaceutical composition may have a pH in the range of about 7.0 to about 7.8. Preferably, the pharmaceutical composition may have a pH in the range of about 5.6 to about 7.6 or about 7.2 to about 7.6, such as 7.4. In some embodiments the pharmaceutical composition has a pH of about 5.6 to about 7.6. In some embodiments the pharmaceutical composition has a pH of about 7.2 to about 7.6. In some embodiments the pharmaceutical composition has a pH of about 7.4. In some embodiments the pharmaceutical composition has a pH of at least 5.6, at least 5.7, at least 5.8, at least 5.9, at least 6.0, at least 6.1, at least 6.2, at least 6.3, at least 6.4, at least 6.5, at least 6.6, at least 6.7, at least 6.8, at least 6.9, at least 7.0, at least 7.1 or at least 7.2. In some embodiments the pharmaceutical composition has a pH of no more than 9.5, no more than 9.4, no more than 9.3, no more than 9.2, no more than 9.1, no more than 9.0, no more than 8.9, no more than 8.8, no more than 8.7, no more than 8.6, no more than 8.5, no more than 8.4, no more than 8.3, no more than 8.2, no more than 8.1, no more than 8.0, no more than 7.9, no more than 7.8, no more than 7.7, no more than 7.6, no more than 7.5, no more than 7.4, no more than 7.3, no more than 7.2, no more than 7.1, no more than 7.0, no more than 6.9, no more than 6.8, no more than 6.7, no more than 6.6, no more than 6.5, no more than 6.4, no more than 6.3, no more than 6.2, no more than 6.1, or no more than 6.0.

In some embodiments the pharmaceutical composition may comprise a buffer. In some embodiments the pharmaceutical composition comprises a phosphate buffer, such as a sodium phosphate buffer, e.g. disodium phosphate. In some embodiments the pharmaceutical composition may comprise an isotonic agent. In some embodiments the isotonic agent is propylene glycol or sodium chloride. The pharmaceutical composition may comprise sodium chloride. In some embodiments the pharmaceutical composition comprises a preservative, such as phenol.

In some embodiments the pharmaceutical composition does not comprise cyclodextrin. In some embodiments the pharmaceutical composition does not comprise a preservative.

The pharmaceutical composition may be in the form of a solution or a suspension. Preferably, the pharmaceutical composition may be in the form of a solution. In some embodiments the pharmaceutical composition is aqueous composition, such as an aqueous solution or an aqueous suspension. The pharmaceutical composition may be in the form of an aqueous solution. The term “aqueous composition” may be defined as a composition comprising water, such as at least 50% (w/w) water. Likewise, the term “aqueous solution” may be defined as a solution comprising at least 50% (w/w) water, and the term “aqueous suspension” is defined as a suspension comprising at least 50% (w/w) water. In some embodiments an aqueous composition comprises at least 50% (w/w) water, such as at least 60% (w/w) or at least 70% (w/w) water. In some embodiments an aqueous composition comprises at least 80% (w/w) or at least 90% (w/w) water.

In some embodiments semaglutide is administered in the form of a pharmaceutical composition comprising a phosphate buffer and propylene glycol. In some embodiments semaglutide is administered in the form of a pharmaceutical composition comprising about 2-15 mM phosphate buffer and about 2-25 mg/ml propylene glycol. In some embodiments semaglutide is administered in the form of a pharmaceutical composition comprising about 0.1-20 mg/ml semaglutide, about 2-15 mM phosphate buffer, about 2-25 mg/ml propylene glycol, and a pH in the range of about 7.0-9.0. In some embodiments semaglutide is administered in the form of a pharmaceutical composition comprising about 0.1-20 mg/ml semaglutide, about 2-15 mM phosphate buffer, about 2-25 mg/ml propylene glycol, about 1-18 mg/ml phenol, and a pH in the range of about 7.0-9.0. In some embodiments semaglutide is administered in the form of a pharmaceutical composition comprising about 1.0 mg/ml semaglutide, about 1.42 mg/ml disodium phosphate dihydrate, about 14.0 mg/ml propylene glycol, about 5.5 mg/ml phenol, and a pH of about 7.4. In some embodiments semaglutide is administered in the form of a pharmaceutical composition comprising about 0.5-4 mg/ml semaglutide, about 1.42 mg/ml disodium phosphate dihydrate, about 14.0 mg/ml propylene glycol, about 5.5 mg/ml phenol, and a pH of about 7.4. In some embodiments semaglutide is administered in the form of a pharmaceutical composition comprising about 0.5-1.5 mg/ml semaglutide, about 1.42 mg/ml disodium phosphate dihydrate, about 14.0 mg/ml propylene glycol, about 5.5 mg/ml phenol, and a pH of about 7.4. In some embodiments semaglutide is administered in the form of a pharmaceutical composition comprising about 1.0-3.5 mg/ml semaglutide, about 1.42 mg/ml disodium phosphate dihydrate, about 14.0 mg/ml propylene glycol, about 5.5 mg/ml phenol, and a pH of about 7.4.

In some embodiments semaglutide is administered in the form of a pharmaceutical composition comprising a phosphate buffer and sodium chloride. In some embodiments semaglutide is administered in the form of a pharmaceutical composition comprising about 2-15 mM phosphate buffer and about 2-25 mg/ml sodium chloride. In some embodiments semaglutide is administered in the form of a pharmaceutical composition comprising about 0.1-20 mg/ml semaglutide, about 2-15 mM phosphate buffer, about 2-25 mg/ml sodium chloride, and a pH in the range of about 7.0-9.0. In some embodiments semaglutide is administered in the form of a pharmaceutical composition comprising about 0.1-20 mg/ml semaglutide, about 2-15 mM phosphate buffer, about 2-25 mg/ml sodium chloride, about 1-18 mg/ml phenol, and a pH in the range of about 7.0-9.0. In some embodiments semaglutide is administered in the form of a pharmaceutical composition comprising about 1.0 mg/ml semaglutide, about 1.42 mg/ml disodium phosphate dihydrate, about 14.0 mg/ml sodium chloride, about 5.5 mg/ml phenol, and a pH of about 7.4. In some embodiments semaglutide is administered in the form of a pharmaceutical composition comprising about 0.5-4 mg/ml semaglutide, about 1.42 mg/ml disodium phosphate dihydrate, about 14.0 mg/ml sodium chloride, about 5.5 mg/ml phenol, and a pH of about 7.4. In some embodiments semaglutide is administered in the form of a pharmaceutical composition comprising about 0.5-1.5 mg/ml semaglutide, about 1.42 mg/ml disodium phosphate dihydrate, about 14.0 mg/ml sodium chloride, about 5.5 mg/ml phenol, and a pH of about 7.4. In some embodiments semaglutide is administered in the form of a pharmaceutical composition comprising about 1.0-3.5 mg/ml semaglutide, about 1.42 mg/ml disodium phosphate dihydrate, about 14.0 mg/ml sodium chloride, about 5.5 mg/ml phenol, and a pH of about 7.4.

In some embodiments the pharmaceutical composition comprises semaglutide and cagrilintide. Semaglutide and cagrilintide may be administered in the form of the pharmaceutical compositions described in WO2021/144476. Semaglutide and cagrilintide may be administered in the form of the pharmaceutical composition described in WO2023/110833. Semaglutide and cagrilintide may be administered in the form of the pharmaceutical composition described in WO2024/256632.

The pharmaceutical composition comprising semaglutide and cagrilintide may preferably have a pH in the range of about 5.6-6.2, such as 5.6-6.0 or 5.8-6.2.

The concentration of cagrilintide in the pharmaceutical composition disclosed herein may be from about 0.25 mg/ml to about 22 mg/ml. The concentration of the amylin receptor agonist may be at least about 0.25 mg/ml. The concentration of the amylin receptor agonist may be, at the most, about 22 mg/ml. The concentration of the amylin receptor agonist may be such as to provide any one of the dosages specified herein.

### Methods of Manufacture

The GLP-1 receptor agonist and/or amylin receptor agonist may be produced by classical peptide synthesis, e.g. solid phase peptide synthesis using t-Boc or Fmoc chemistry, or other well established techniques, such as those described in Greene and Wuts, “Protective Groups in Organic Synthesis”, John Wiley & Sons, 1999; Florencio Zaragoza Dörwald, “Organic Synthesis on Solid Phase”, Wiley-VCH Verlag GmbH, 2000; and “Fmoc Solid Phase Peptide Synthesis”, Edited by W. C. Chan and P. D. White, Oxford University Press, 2000.

Alternatively, the GLP-1 receptor agonist and/or amylin receptor agonist may be produced by recombinant expression techniques, e.g. by culturing a host cell containing a DNA sequence encoding the peptide sequence and capable of expressing the peptide, in a suitable nutrient medium under conditions permitting the expression of the peptide. Non-limiting examples of host cells suitable for expression of these peptides are Escherichia coli, Saccharomyces cerevisiae and mammalian BHK or CHO cell lines.

GLP-1 receptor agonists comprising one or more non-proteogenic amino acids may also be produced, semi-synthetically, using a combination of recombinant expression techniques and chemical peptide synthesis as described in WO2009/083549. Compounds which comprise one or more non-natural amino acids and/or a covalently attached N-terminal mono- or dipeptide mimetic may also be produced as described in Hodgson et al in “The synthesis of peptides and proteins containing non-natural amino acids”, Chemical Society Reviews, vol. 33, no. 7 (2004), p. 422-430.

Once the GLP-1 receptor agonist and/or amylin receptor agonist have been manufactured and purified, the pharmaceutical composition disclosed herein may be prepared using the method described in WO2023/187067.

Alternatively, or subsequently, the composition comprising the drug substances may be freeze- or spray-dried using methods known to the person skilled in the art. Ohtake, S., Izutsu, K. I., & Lechuga-Ballesteros, D. (Eds.). describe such methods in “Drying technologies for biotechnology and pharmaceutical applications” (2020) John Wiley & Sons. The composition comprising the drug substances and cyclodextrin that is dried may further comprise a surfactant, such as polysorbate 20 and/or 80.

If the composition is intermediately freeze- or spray-dried then the dry formulation may be dissolved or “reconstituted” in aqueous solution prior to use. Said reconstituted aqueous solution may be presented in a vial. Said reconstituted aqueous solution may comprise or consist of a pre-determined amount of water for injection. Said reconstituted aqueous solution may comprise or consist of a pre-determined amount of water for injection and one or more preservatives. Said reconstituted aqueous solution may comprise or consist of a pre-determined amount of water for injection and phenol and/or m-cresol and/or EDTA. Said reconstituted aqueous solution may comprise or consist of a pre-determined amount of water for injection, phenol and/or EDTA. Said reconstituted aqueous solution may comprise or consist of a pre-determined amount of water for injection and m-cresol and/or EDTA. Said reconstituted aqueous solution may comprise or consist of a pre-determined amount of water for injection and phenol and/or m-cresol. Said reconstituted aqueous solution may comprise or consist of a pre-determined amount of water for injection and phenol. Said reconstituted aqueous solution may comprise or consist of a pre-determined amount of water for injection and m-cresol. Said reconstituted aqueous solution may comprise or consist of a pre-determined amount of water for injection and EDTA. Said reconstituted aqueous solution may further comprise a buffer having at least one pKa value of about 5.0-7.0, such as histidine or citrate. Said reconstituted aqueous solution may further comprise sorbitol.

Unless otherwise stated, ranges herein include their end points. In some embodiments the term “a” means “one or more”. In some embodiments, and unless otherwise indicated herein, terms presented in singular form also include the plural situation. Herein the terms “about” and “approximately” may be understood to permit standard variation as would be understood by those of ordinary skill in the art. In some embodiments, the term “about” means±10% of the value referred to, and includes the value. In some embodiments the term “comprise” as used herein includes “consist of”. In some embodiments, pH values referred to herein are measured at 25° C. Semaglutide 2.4 mg may be referred to as “semaglutide 2.4” herein.

### Non-Limiting Embodiments of the Invention

The invention is further described by the following non-limiting embodiments:
1. A method for weight management of a human subject in need thereof comprising subcutaneous administration of up to about 7.2 mg semaglutide per week.
2. A method for weight management of a human subject in need thereof comprising subcutaneous administration of about 6.0 to about 7.2 mg semaglutide per week.
3. A method for weight management of a human subject in need thereof comprising subcutaneous administration of about 7.2 mg semaglutide per week.
4. A method for weight management of a human subject in need thereof comprising subcutaneous administration of about 7.2 mg semaglutide once weekly.
5. A method for treatment of obesity or overweight comprising subcutaneous administration of about 7.2 mg semaglutide once weekly to a subject in need thereof.
6. A method for treatment of obesity comprising subcutaneous administration of about 7.2 mg semaglutide once weekly to a subject in need thereof.
7. A method for treatment of overweight comprising subcutaneous administration of about 7.2 mg semaglutide once weekly to a subject in need thereof.
8. A method for treatment of overweight comprising subcutaneous administration of about 7.2 mg semaglutide once weekly to a subject in need thereof, wherein said subject further has at least one weight-related co-morbidity.
9. The method according to any one of the preceding embodiments, wherein said subject has obesity or overweight.
10. The method according to any one of the preceding embodiments, wherein said subject has obesity.
11. The method according to any one of the preceding embodiments, wherein said subject has overweight.
12. The method according to any one of the preceding embodiments, wherein said subject has overweight and at least one weight-related co-morbidity.
13. The method according to any one of the preceding embodiments, wherein said weight-related co-morbidity is selected from the group consisting of diabetes, cardiovascular disease, metabolic dysfunction-associated steatohepatitis, alcoholic liver disease, and obstructive sleep apnoea.
14. The method according to any one of the preceding embodiments, wherein said subject has a BMI of at least 30 kg/m².
15. The method according to any one of the preceding embodiments, wherein said subject has a BMI of at least 35 kg/m².
16. The method according to any one of the preceding embodiments, wherein said subject has a BMI of at least 40 kg/m².
17. The method according to any one of the preceding embodiments, wherein said subject has a BMI of 30-50 kg/m².
18. The method according to any one of the preceding embodiments, wherein said subject has a BMI of 30-45 kg/m².
19. The method according to any one of the preceding embodiments, wherein said subject has a BMI of 30-40 kg/m².
20. The method according to any one of the preceding embodiments, wherein said subject has a BMI of 30-35 kg/m².
21. The method according to any one of the preceding embodiments, wherein said subject has a BMI of at least 27 kg/m².
22. The method according to any one of the preceding embodiments, wherein said subject has a BMI of 27-30 kg/m².
23. The method according to any one of the preceding embodiments, wherein said subject has a BMI of at least 30 kg/m² and a body weight of at least about 100 kg.
24. The method according to any one of the preceding embodiments, wherein said subject has a BMI of at least 30 kg/m² and a body weight of at least about 120 kg.
25. The method according to any one of the preceding embodiments, wherein said subject has a BMI of at least 30 kg/m² and a body weight of at least about 140 kg.
26. The method according to any one of the preceding embodiments, wherein said subject is adult or paediatric.
27. The method according to any one of the preceding embodiments, wherein said subject is adult.
28. The method according to any one of the preceding embodiments, wherein said subject is paediatric.
29. The method according to any one of the preceding embodiments, wherein said subject is paediatric aged 12 years or older.
30. The method according to any one of the preceding embodiments, wherein said subject is paediatric and with a BMI corresponding to of least 30 kg/m² for adults.
31. The method according to any one of the preceding embodiments, wherein said subject has diabetes.
32. The method according to any one of the preceding embodiments, wherein said subject has diabetes, such as type 2 diabetes.
33. The method according to any one of the preceding embodiments, wherein said subject has at least one weight-related comorbid condition, such as dyslipidemia or hypertension.
34. The method according to any one of the preceding embodiments, wherein said subject has overweight and at least one weight-related comorbid condition, such as dyslipidemia or hypertension.
35. The method according to any one of the preceding embodiments, wherein said subject has a BMI of at least 27 kg/m² and at least one weight-related comorbid condition, such as dyslipidemia or hypertension.
36. The method according to any one of the preceding embodiments, wherein said subject is not classified as New York Heart Association (NYHA) class IV.
37. The method according to any one of the preceding embodiments, wherein said semaglutide is administered in an amount of 7.2 mg semaglutide per week.
38. The method according to any one of the preceding embodiments, wherein said semaglutide is administered once weekly.
39. The method according to any one of the preceding embodiments, wherein said semaglutide is administered in an amount of about 7.2 mg semaglutide once weekly.
40. The method according to any one of the preceding embodiments, wherein said semaglutide is administered in an amount of 7.2 mg semaglutide once weekly.
41. The method according to any one of the preceding embodiments, wherein said semaglutide is administered for at least 24 weeks.
42. The method according to any one of the preceding embodiments, wherein said semaglutide is administered for at least 32 weeks.
43. The method according to any one of the preceding embodiments, wherein said semaglutide is administered for at least 40 weeks.
44. The method according to any one of the preceding embodiments, wherein said semaglutide is administered for at least about 52 weeks.
45. The method according to any one of the preceding embodiments, wherein said semaglutide is administered for at least about 72 weeks.
46. The method according to any one of the preceding embodiments, wherein said semaglutide is administered for about 72 weeks.
47. The method according to any one of the preceding embodiments, wherein said semaglutide is administered for at least about 72 weeks from the initial semaglutide dosage escalation step.
48. The method according to any one of the preceding embodiments, wherein said semaglutide is administered for up to about 72 weeks from the initial semaglutide dosage escalation step.
49. The method according to any one of the preceding embodiments, wherein said semaglutide is administered for as at least about 72 weeks including dosage escalation.
50. The method according to any one of the preceding embodiments, wherein said semaglutide is administered for as about 72 weeks including dosage escalation.
51. The method according to any one of the preceding embodiments, wherein said semaglutide is administered for at least about 52 weeks of said of about 7.2 mg semaglutide per week.
52. The method according to any one of the preceding embodiments, wherein said subcutaneous administration further comprises one or more dosage escalation steps of up to about 2.4 mg semaglutide per week followed by a dosage of said about 7.2 mg semaglutide per week.
53. The method according to any one of the preceding embodiments, wherein said subcutaneous administration further comprises one or more dosage escalation steps of about 0.25 mg to about 2.4 mg semaglutide per week followed by a dosage of said about 7.2 mg semaglutide per week.
54. The method according to any one of the preceding embodiments, wherein said subcutaneous administration comprises a dosage escalation step of about 2.4 mg semaglutide per week followed by a dosage of said about 7.2 mg semaglutide per week.
55. The method according to any one of the preceding embodiments, wherein said method further comprises one or more dosage escalation steps of administration of up to about 2.4 mg semaglutide per week, prior to administration of said about 7.2 mg semaglutide per week.
56. The method according to any one of the preceding embodiments, wherein said administration comprises a dosage escalation step of administration of about 2.4 mg semaglutide per week, prior to administration of said about 7.2 mg semaglutide per week.
57. The method according to any one of the preceding embodiments, wherein said subcutaneous administration comprises dosage escalation steps for up to about 20 weeks, such as for about 20 weeks.
58. The method according to any one of the preceding embodiments, wherein said subcutaneous administration comprises dosage escalation steps for up to about 20 weeks at no more than about 2.4 mg semaglutide per week followed by a dosage of said about 7.2 mg semaglutide per week.
59. The method according to any one of the preceding embodiments, wherein said subcutaneous administration comprises dosage escalation steps every about four weeks from about 0.25 mg semaglutide per week, to about 0.5 mg semaglutide per week, to about 1.0 mg semaglutide per week, to about 1.7 mg semaglutide per week, to about 2.4 mg semaglutide per week followed by a dosage of said about 7.2 mg semaglutide per week.
60. The method according to any one of the preceding embodiments, wherein said method comprises a dosage escalation step at about 2.4 mg semaglutide per week one week prior to the first administration of about 7.2 mg semaglutide per week.
61. The method according to any one of the preceding embodiments, wherein said method comprises a dosage escalation step at 2.4 mg semaglutide once weekly one week prior to the first administration of 7.2 mg semaglutide once weekly.
62. The method according to any one of the preceding embodiments, wherein said method comprises a dosage escalation steps from weekly semaglutide administration in an amount of about (i) 0.25 mg to (ii) 0.5 mg to (iii) 1.0 mg to (iv) 1.7 mg to (v) 2.4 mg prior to administration of said about 7.2 mg semaglutide per week.
63. The method according to any one of the preceding embodiments, wherein said method comprises a dosage escalation steps from once weekly semaglutide administration in an amount of about (i) 0.25 mg to (ii) 0.5 mg to (iii) 1.0 mg to (iv) 1.7 mg to (v) 2.4 mg prior to administration of said about 7.2 mg semaglutide once weekly.
64. The method according to any one of the preceding embodiments, wherein said method comprises a dosage escalation steps about every four weeks from once weekly semaglutide administration in an amount of about (i) 0.25 mg to (ii) 0.5 mg to (iii) 1.0 mg to (iv) 1.7 mg to (v) 2.4 mg prior to administration of said about 7.2 mg semaglutide once weekly.
65. The method according to any one of the preceding embodiments, wherein said method comprises a dosage escalation steps every four weeks from once weekly semaglutide administration in an amount of (i) 0.25 mg to (ii) 0.5 mg to (iii) 1.0 mg to (iv) 1.7 mg to (v) 2.4 mg prior to administration of said 7.2 mg semaglutide once weekly.
66. The method according to any one of the preceding embodiments, wherein said method provides a body weight reduction of said subject.
67. The method according to any one of the preceding embodiments, wherein said body weight reduction is at least about 15%, such as at least 18% or at least 20%.
68. The method according to any one of the preceding embodiments, wherein said semaglutide provides a body weight reduction of at least about 15%, such as at least 18% or at least 20%.
69. The method according to any one of the preceding embodiments, wherein said body weight reduction is at least about 15%.
70. The method according to any one of the preceding embodiments, wherein said body weight reduction is at least 18%.
71. The method according to any one of the preceding embodiments, wherein said body weight reduction is at least 20%.
72. The method according to any one of the preceding embodiments, wherein said body weight reduction is at least 25%.
73. The method according to any one of the preceding embodiments, wherein semaglutide provides a body weight reduction of at least about 15%, such as at least 18% or at least 20%.
74. The method according to any one of the preceding embodiments, wherein said about 7.2 mg semaglutide per week provides a body weight reduction of at least about 15%, such as at least 18% or at least 20%.
75. The method according to any one of the preceding embodiments, wherein said about 7.2 mg semaglutide per week administered for at least 52 weeks, such as at least about 72 weeks including dosage escalation, provides a body weight reduction of at least about 15%, such as at least 18% or at least 20%.
76. The method according to any one of the preceding embodiments, wherein said about 7.2 mg semaglutide per week administered for at least 52 weeks provides a body weight reduction of at least about 15%, such as at least 18% or at least 20%.
77. The method according to any one of the preceding embodiments, wherein said about 7.2 mg semaglutide per week administered for at least about 72 weeks including dosage escalation, provides a body weight reduction of at least about 15%, such as at least 18% or at least 20%.
78. The method according to any one of the preceding embodiments, wherein said about 7.2 mg semaglutide per week including administered for at least 52 weeks provides a body weight reduction of at least about 15%.
79. The method according to any one of the preceding embodiments, wherein said about 7.2 mg semaglutide per week including administered for at least 52 weeks provides a body weight reduction of at least about 20%.
80. The method according to any one of the preceding embodiments, wherein said about 7.2 mg semaglutide per week including administered for at least 52 weeks provides a body weight reduction of at least about 25%.
81. The method according to any one of the preceding embodiments, wherein said body weight reduction is at least about 15% after at least about 52 weeks of administration of said about 7.2 mg semaglutide.
82. The method according to any one of the preceding embodiments, wherein said body weight reduction is about 15% after about 52 weeks of administration of said about 7.2 mg semaglutide.
83. The method according to any one of the preceding embodiments, wherein said body weight reduction is at least 15% after about 52 weeks of administration of said 7.2 mg semaglutide.
84. The method according to any one of the preceding embodiments, wherein said body weight reduction is at least 18% after about 52 weeks of administration of said 7.2 mg semaglutide.
85. The method according to any one of the preceding embodiments, wherein said body weight reduction is at least 20% after at least about 52 weeks of administration of said about 7.2 mg semaglutide.
86. The method according to any one of the preceding embodiments, wherein said body weight reduction is at least 25% after at least about 52 weeks of administration of said about 7.2 mg semaglutide.
87. The method according to any one of the preceding embodiments, wherein said body weight reduction is by up to 75%.
88. The method according to any one of the preceding embodiments, wherein said body weight reduction is by up to 75% after at least about 52 weeks of administration of said about 7.2 mg semaglutide.
89. The method according to any one of the preceding embodiments, wherein a greater proportion of subjects obtain a body weight reduction of at least 20% is greater by administration of said 7.2 mg semaglutide once weekly compared to 2.4 mg semaglutide once weekly.
90. The method according to any one of the preceding embodiments, wherein at least 45%, such as about 50%, of subjects obtain a body weight reduction of at least 20% with said 7.2 mg semaglutide.
91. The method according to any one of the preceding embodiments, wherein a greater proportion of subjects obtain a body weight reduction of at least 25% is greater by administration of said about 7.2 mg semaglutide once weekly compared to 2.4 mg semaglutide once weekly.
92. The method according to any one of the preceding embodiments, wherein at least 25%, such as at least 30%, such as at least about 33%, of subjects obtain a body weight reduction of at least 25% with said about 7.2 mg semaglutide.
93. The method according to any one of the preceding embodiments, wherein a greater proportion of subjects obtain a BMI of less than 27 kg/m².
94. The method according to any one of the preceding embodiments, wherein a greater proportion of subjects obtain a BMI of less than 27 kg/m² with said administration of about 7.2 mg semaglutide per week compared to about 2.4 mg semaglutide per week.
95. The method according to any one of the preceding embodiments, wherein a greater proportion of subjects obtain a BMI of less than 27 kg/m² with said administration of 7.2 mg semaglutide once weekly compared to 2.4 mg semaglutide once weekly.
96. The method according to any one of the preceding embodiments, wherein at least about 25%, such as about 27%, of subjects obtain a BMI of less than 27 kg/m² with said 7.2 mg semaglutide.
97. The method according to any one of the preceding embodiments, wherein a greater proportion of subjects obtain a BMI of less than 27 kg/m² with said administration of about 7.2 mg semaglutide per week compared to about 2.4 mg semaglutide per week.
98. The method according to any one of the preceding embodiments, wherein a greater proportion of subjects obtain a BMI of less than 27 kg/m² with said administration of 7.2 mg semaglutide once weekly compared to 2.4 mg semaglutide once weekly.
99. The method according to any one of the preceding embodiments, wherein said method provides a reduction of lean body volume compared to total fat volume of less than 20%, such as less than 18% or less than 16%.
100. The method according to any one of the preceding embodiments, wherein said method provides a reduction of lean body volume compared to total fat volume of no more than 15%.
101. The method according to any one of the preceding embodiments, wherein said method provides a reduction of lean body volume compared to total fat volume of about 10% to about 20%.
102. The method according to any one of the preceding embodiments, wherein said method is tolerable for said subject.
103. The method according to any one of the preceding embodiments, wherein less than about 15% of subjects discontinue administration of said about 7.2 mg semaglutide.
104. The method according to any one of the preceding embodiments, wherein about 8% to about 15% of subjects discontinue administration of said about 7.2 mg semaglutide.
105. The method according to any one of the preceding embodiments, wherein about 8% to about 15% of subjects discontinue administration of said about 7.2 mg semaglutide.
106. The method according to any one of the preceding embodiments, wherein less than about 15% of subjects discontinue administration of said about 7.2 mg semaglutide within at least about 52 weeks.
107. The method according to any one of the preceding embodiments, wherein less than 15% of subjects discontinue administration of said about 7.2 mg semaglutide within at least about 52 weeks after an initial dosage escalation, wherein said dosage escalation is as defined herein.
108. The method according to any one of the preceding embodiments, wherein less than 15% of subjects discontinue administration of said about 7.2 mg semaglutide within at least about 52 weeks after an initial dosage escalation of up to about 2.4 mg semaglutide once weekly.
109. The method according to any one of the preceding embodiments, wherein less than 15% of subjects discontinue administration of said about 7.2 mg semaglutide within up to about 72 weeks from the initial semaglutide dosage escalation step, such as an initial semaglutide dosage escalation step of 0.25 mg semaglutide once weekly.
110. The method according to any one of the preceding embodiments, wherein less than 15% of subjects discontinue administration of said about 7.2 mg semaglutide within up to about 72 weeks from the initial semaglutide dosage escalation step, such as an initial semaglutide dosage escalation step of 0.25 mg semaglutide once weekly, and said method comprising dosage escalation of up to about 2.4 mg semaglutide once weekly.
111. The method according to any one of the preceding embodiments, wherein less than 12% of subjects discontinue administration of said about 7.2 mg semaglutide.
112. The method according to any one of the preceding embodiments, wherein less than 12% of subjects discontinue administration of said about 7.2 mg semaglutide within at least about 52 weeks.
113. The method according to any one of the preceding embodiments, wherein less than 12% of subjects discontinue administration of said about 7.2 mg semaglutide within at least about 52 weeks after an initial dosage escalation, wherein said dosage escalation is as defined herein.
114. The method according to any one of the preceding embodiments, wherein less than 12% of subjects discontinue administration of said about 7.2 mg semaglutide within at least about 52 weeks after an initial dosage escalation of up to about 2.4 mg semaglutide once weekly.
115. The method according to any one of the preceding embodiments, wherein less than 12% of subjects discontinue administration of said about 7.2 mg semaglutide within up to about 72 weeks from the initial semaglutide dosage escalation step, such as an initial semaglutide dosage escalation step of 0.25 mg semaglutide once weekly.
116. The method according to any one of the preceding embodiments, wherein less than 12% of subjects discontinue administration of said about 7.2 mg semaglutide within up to about 72 weeks from the initial semaglutide dosage escalation step, such as an initial semaglutide dosage escalation step of 0.25 mg semaglutide once weekly, and said method comprising dosage escalation of up to about 2.4 mg semaglutide once weekly.
117. The method according to any one of the preceding embodiments, wherein said discontinue is a permanent discontinuation, such as a discontinuation for at least 4 weeks or at least 8 weeks.
118. The method according to any one of the preceding embodiments, wherein less than about 15% of subjects discontinue administration of said about 7.2 mg semaglutide and said semaglutide provides a body weight reduction of at least about 15%.
119. The method according to any one of the preceding embodiments, wherein less than about 15% of subjects discontinue administration of said about 7.2 mg semaglutide and said semaglutide provides a body weight reduction of at least about 15%.
120. The method according to any one of the preceding embodiments, wherein less than about 15% of subjects discontinue administration of said about 7.2 mg semaglutide and said semaglutide provides a body weight reduction of at least about 15%.
121. The method according to any one of the preceding embodiments, wherein about 65% to about 75% of subjects have at least one gastrointestinal adverse event.
122. The method according to any one of the preceding embodiments, wherein about 65% to about 75% of subjects have at least one gastrointestinal adverse event during administration of said about 7.2 mg semaglutide within about 52 weeks.
123. The method according to any one of the preceding embodiments, wherein about 71% % of subjects have at least one gastrointestinal adverse event during administration of said about 7.2 mg semaglutide within about 52 weeks.
124 The method according to any one of the preceding embodiments, wherein about 71% % of subjects have at least one gastrointestinal adverse event during administration of said about 7.2 mg semaglutide within about 72 weeks including dosage escalation.
125. The method according to any one of the preceding embodiments, wherein about 65% to about 75% of subjects have at least one gastrointestinal adverse event.
126. The method according to any one of the preceding embodiments, wherein about 35% to about 50% of subjects have at least one gastrointestinal adverse event.
127. The method according to any one of the preceding embodiments, wherein about 40% to about 48% of subjects have at least one gastrointestinal adverse event.
128. The method according to any one of the preceding embodiments, wherein about 44% of subjects have at least one gastrointestinal adverse event.
129. The method according to any one of the preceding embodiments, wherein the waist circumference of said subject is reduced.
130. The method according to any one of the preceding embodiments, wherein the blood pressure of said subject is reduced.
131. The method according to any one of the preceding embodiments, wherein the diastolic blood pressure and/or systolic blood pressure of said subject is reduced.
132. The method according to any one of the preceding embodiments, wherein the diastolic blood pressure and systolic blood pressure of said subject is reduced.
133. The method according to any one of the preceding embodiments, wherein said semaglutide is administered via subcutaneous injection.
134. The method according to any one of the preceding embodiments, wherein said semaglutide is administered in the form of a pharmaceutical composition comprising semaglutide and one or more excipients.
135. The method according to any one of the preceding embodiments, wherein said semaglutide is administered in the form of a pharmaceutical composition consisting essentially of semaglutide and one or more excipients.
136. The method according to any one of the preceding embodiments, wherein said semaglutide is administered in the form of a pharmaceutical composition consisting of semaglutide and one or more excipients.
137. The method according to any one of the preceding embodiments, wherein said pharmaceutical composition is a solution.
138. The method according to any one of the preceding embodiments, wherein said pharmaceutical composition is an aqueous solution.
139. The method according to any one of the preceding embodiments, wherein said semaglutide is administered in the form of a pharmaceutical composition consisting of semaglutide and one or more excipients and said composition has a pH in the range of about 5.5 to about 10.0.
140. The method according to any one of the preceding embodiments, wherein said pharmaceutical composition has a pH in the range of about 5.6 to about 10.0.
141. The method according to any one of the preceding embodiments, wherein said pharmaceutical composition has a pH in the range of about 6.0 to about 9.0.
142. The method according to any one of the preceding embodiments, wherein said pharmaceutical composition has a pH in the range of about 6.8 to about 8.0.
143. The method according to any one of the preceding embodiments, wherein said pharmaceutical composition has a pH in the range of about 7.0 to about 7.8.
144. The method according to any one of the preceding embodiments, wherein said pharmaceutical composition has a pH in the range of about 7.2 to about 7.6.
145. The method according to any one of the preceding embodiments, wherein said pharmaceutical composition has a pH of about 7.4.
146. The method according to any one of the preceding embodiments, wherein said pharmaceutical composition comprises a buffer.
147. The method according to any one of the preceding embodiments, wherein said pharmaceutical composition comprises a phosphate buffer.
148. The method according to any one of the preceding embodiments, wherein said pharmaceutical composition comprises an isotonic agent.
149. The method according to any one of the preceding embodiments, wherein said pharmaceutical composition comprises sodium chloride.
150. The method according to any one of the preceding embodiments, wherein said pharmaceutical composition does not comprise cyclodextrin.
151. The method according to any one of the preceding embodiments, wherein said pharmaceutical composition does not comprise a preservative.
152. The method according to any one of the preceding embodiments, wherein said pharmaceutical composition comprises a preservative.
153. The method according to any one of the preceding embodiments, wherein said semaglutide is contained in a vial.
154. The method according to any one of the preceding embodiments, wherein said semaglutide is contained in an injection device.
155. The method according to any one of the preceding embodiments, wherein said semaglutide is administered via an injection device.
156. A method for treating or reducing the risk of cardiovascular disease in a human subject in need thereof comprising subcutaneous administration of about 7.2 mg semaglutide per week.
157. The method according to the preceding embodiment wherein said method is as defined in any one of embodiments 9-155.
158. A method for reducing the risk of major adverse cardiovascular event comprising CV death, non-fatal myocardial infarction and non-fatal stroke in a human subject in need thereof comprising subcutaneous administration of about 7.2 mg semaglutide per week.
159. The method according to the preceding embodiment wherein said method is as defined in any one of embodiments 9-155.
160. A method for treating or reducing the risk of heart failure in a human subject in need thereof comprising subcutaneous administration of about 7.2 mg semaglutide per week.
161. The method according to the preceding embodiment wherein said method is as defined in any one of embodiments 9-155.
162. A method for treating metabolic dysfunction-associated steatohepatitis (MASH) in a human subject in need thereof comprising subcutaneous administration of about 7.2 mg semaglutide per week.
163. The method according to the preceding embodiment wherein said method is as defined in any one of embodiments 9-155.
164. A method for treatment of alcoholic liver disease (ALD) in a human subject in need thereof comprising subcutaneous administration of about 7.2 mg semaglutide per week.
165. The method according to the preceding embodiment wherein said method is as defined in any one of embodiments 9-155.
166. A method for treatment of type 2 diabetes in a human subject in need thereof comprising subcutaneous administration of about 7.2 mg semaglutide per week.
167. The method according to the preceding embodiment wherein said method is as defined in any one of embodiments 9-155.
168. A method for treatment of Alzheimer's disease in a human subject in need thereof comprising subcutaneous administration of about 7.2 mg semaglutide per week.
169. The method according to the preceding embodiment wherein said method is as defined in any one of embodiments 9-155.
170. A method for treatment of chronic kidney disease in a human subject in need thereof comprising subcutaneous administration of about 7.2 mg semaglutide per week.
171. The method according to the preceding embodiment wherein said method is as defined in any one of embodiments 9-155.
172. A method for treatment of peripheral arterial disease in a human subject in need thereof comprising subcutaneous administration of about 7.2 mg semaglutide per week.
173. The method according to the preceding embodiment wherein said method is as defined in any one of embodiments 9-155.
174. A method for treatment of obstructive sleep apnoea in a human subject in need thereof, such as a subject with overweight or obesity, comprising subcutaneous administration of about 7.2 mg semaglutide per week.
175. The method according to any one of the preceding embodiments, wherein said Alzheimer's disease is early Alzheimer's disease.
176. The method according to any one of the preceding embodiments, wherein said heart failure is heart failure with preserved ejection fraction.
177. The method according to any one of the preceding embodiments, wherein said method comprises administration of a further active ingredient.
178. The method according to any one of the preceding embodiments, wherein said method further comprises administration of cagrilintide.
179. The method according to any one of the preceding embodiments, wherein said cagrilintide is administered simultaneously with or sequentially to said semaglutide.
180. The method according to any one of the preceding embodiments, wherein said cagrilintide is administered via subcutaneous administration.
181. The method according to any one of the preceding embodiments, wherein said cagrilintide is administered via subcutaneous injection.
182. The method according to any one of the preceding embodiments, wherein said cagrilintide is administered in amount of about 0.25-16 mg per week.
183. The method according to any one of the preceding embodiments, wherein said cagrilintide is administered in amount of about 0.25-9.0 mg per week.
184. The method according to any one of the preceding embodiments, wherein said cagrilintide is administered in amount of about 0.25-4.5 mg per week.
185.
186. The method according to any one of the preceding embodiments, wherein said cagrilintide is administered in amount of about 0.25 mg per week.
187. The method according to any one of the preceding embodiments, wherein said cagrilintide is administered in amount of 0.5 mg per week.
188. The method according to any one of the preceding embodiments, wherein said cagrilintide is administered in amount of 1.0 mg per week.
189. The method according to any one of the preceding embodiments, wherein said cagrilintide is administered in amount of 1.7 mg per week.
190. The method according to any one of the preceding embodiments, wherein said cagrilintide is administered in amount of 2.4 mg per week.
191. The method according to any one of the preceding embodiments, wherein said cagrilintide is administered in amount of 3.6 mg per week.
192. The method according to any one of the preceding embodiments, wherein said cagrilintide is administered once weekly.
193. The method according to any one of the preceding embodiments, wherein said cagrilintide is administered in amount of about 0.25-16 mg once weekly.
194. The method according to any one of the preceding embodiments, wherein said cagrilintide is administered in amount of about 0.25 mg once weekly.
195. The method according to any one of the preceding embodiments, wherein said cagrilintide is administered in amount of 0.5 mg once weekly.
196. The method according to any one of the preceding embodiments, wherein said cagrilintide is administered in amount of 1.0 mg once weekly.
197. The method according to any one of the preceding embodiments, wherein said cagrilintide is administered in amount of 1.7 mg once weekly.
198. The method according to any one of the preceding embodiments, wherein said cagrilintide is administered in amount of 2.4 mg once weekly.
199. The method according to any one of the preceding embodiments, wherein said cagrilintide is administered in the form of a pharmaceutical composition comprising cagrilintide and one or more excipients.
200. The method according to any one of the preceding embodiments, wherein said cagrilintide is administered in the form of a pharmaceutical composition comprising cagrilintide, semaglutide and one or more excipients.
201. The method according to any one of the preceding embodiments, wherein said semaglutide is administered in the form of a pharmaceutical composition consisting of cagrilintide, semaglutide and one or more excipients.
202. The method according to any one of the preceding embodiments, wherein said pharmaceutical composition comprising cagrilintide is a solution.
203. The method according to any one of the preceding embodiments, wherein said pharmaceutical composition comprising cagrilintide is an aqueous solution.
204. The method according to any one of the preceding embodiments, wherein the pharmaceutical composition comprising cagrilintide and semaglutide has a pH in the range of 5.6-6.0.
205. The method according to any one of the preceding embodiments, wherein the pharmaceutical composition comprising cagrilintide and semaglutide has a pH in the range of 5.8-6.2.
206. The method according to any one of the preceding embodiments, wherein said pharmaceutical composition comprising cagrilintide has a pH of about 3.5-4.5, such as pH 4.0.
207. The method according to any one of the preceding embodiments, wherein said pharmaceutical composition comprising cagrilintide comprises a buffer.
208. The method according to any one of the preceding embodiments, wherein said pharmaceutical composition comprising cagrilintide comprises a glutamic acid, glutamate, lactic acid, lactate, acetic acid, or acetate buffer.
209. The method according to any one of the preceding embodiments, wherein said pharmaceutical composition comprising cagrilintide comprises a glutamic acid or glutamate buffer.
210. The method according to any one of the preceding embodiments, wherein said pharmaceutical composition comprising cagrilintide comprises a lactic acid or lactate buffer.
211. The method according to any one of the preceding embodiments, wherein said pharmaceutical composition comprising cagrilintide comprises an acetic acid or acetate buffer.
212. Semaglutide for use in weight management comprising subcutaneous administration of up to about 7.2 mg semaglutide per week to a human subject.
213. Semaglutide for use in weight management comprising subcutaneous administration of about 7.2 mg semaglutide per week to a human subject.
214. The semaglutide for use according to the preceding embodiment, wherein said subject is overweight or has obesity.
215. Semaglutide for use in the treatment of obesity comprising subcutaneous administration of about 7.2 mg semaglutide per week to a human subject.
216. Semaglutide for use in the treatment of obesity comprising subcutaneous administration of about 7.2 mg semaglutide per week to a human subject.
217. Semaglutide for use according to any of embodiments 212-216, wherein said use is as defined in any one of the methods of the preceding embodiments.
218. Use of semaglutide in the manufacture of a medicament for use in weight management comprising subcutaneous administration of up to about 7.2 mg semaglutide per week to a human subject.
219. Use of semaglutide in the manufacture of a medicament for use in weight management comprising subcutaneous administration of about 7.2 mg semaglutide per week to a human subject.
220. The use according to the preceding embodiment, wherein said subject is overweight or has obesity.
221. Use of semaglutide in the manufacture of a medicament for the treatment of obesity comprising subcutaneous administration of about 7.2 mg semaglutide per week to a human subject.
222. Use of semaglutide in the manufacture of a medicament for the treatment of obesity comprising subcutaneous administration of about 7.2 mg semaglutide per week to a human subject.
223. The use according to any of embodiments 218-222, wherein said use is as defined in any one of the methods of the preceding embodiments.
224. A pharmaceutical composition comprising semaglutide formulated for subcutaneous administration of up to about 7.2 semaglutide per week.
225. A pharmaceutical composition comprising semaglutide formulated for subcutaneous administration of about 7.2 semaglutide per week.
226. A pharmaceutical composition comprising semaglutide formulated for subcutaneous administration of about 7.2 semaglutide per week to a human subject with overweight or obesity.
227. A pharmaceutical composition comprising semaglutide formulated for subcutaneous administration of about 7.2 semaglutide per week to a human subject with obesity.
228. The pharmaceutical composition according to any of embodiments 224-227, further comprising features as defined in any one of the methods of the preceding embodiments.
229. A kit comprising semaglutide and instructions for use wherein said semaglutide is for subcutaneous administration of up to about 7.2 semaglutide per week.
230. A kit comprising semaglutide and instructions for use wherein said semaglutide is for subcutaneous administration of about 7.2 semaglutide per week.
231. The kit according to embodiment 229 or 230, wherein said semaglutide is contained in a vial.
232. The kit according to embodiment 229 or 230, wherein said semaglutide is contained in an injection device.
233. The kit according any one of embodiments 229-232, further comprising features as defined in any one of the methods of the preceding embodiments.
234. A pharmaceutical composition comprising semaglutide, for use in a method for weight management of a human subject in need thereof, wherein the method comprises subcutaneous administration of about 7.2 mg semaglutide per week.
235. The pharmaceutical composition according to embodiment 234, wherein said weight management is the treatment of obesity or overweight, wherein for said treatment of overweight said subject may have at least one weight-related co-morbidity.
236. The pharmaceutical composition according to embodiment 234 or 235, wherein at least 25% of subjects obtain a body weight reduction of at least 25% with said about 7.2 mg semaglutide.
237. The pharmaceutical composition according to any one of embodiments 234-236, wherein said method provides a body weight reduction of at least about 15%.
238. The pharmaceutical composition according to any one of embodiments 234-237, wherein said method provides a body weight reduction of at least 18%.
239. The pharmaceutical composition according to any one of embodiments 234-238, wherein said method provides a reduction of lean body volume compared to total fat volume of less than 20%.
240. The pharmaceutical composition according to any one of embodiments 234-239, wherein said semaglutide is administered for at least about 52 weeks, such as about 72 weeks from the initial semaglutide dosage escalation step.
241. The pharmaceutical composition according to any one of embodiments 234-240, wherein said subcutaneous administration further comprises one or more dosage escalation steps of administration of up to about 2.4 mg semaglutide per week, prior to administration of said about 7.2 mg semaglutide per week.
242. The pharmaceutical composition according to any one of embodiments 234-241, wherein said semaglutide is administered once weekly.
243. The pharmaceutical composition according to any one of embodiments 234-242, wherein said method does not comprise administration of cyclodextrin.
244. The pharmaceutical composition according to any one of embodiments 234-243, wherein said pharmaceutical composition has a pH in the range of about 5.6 to about 9.0.
245. The pharmaceutical composition according to any one of embodiments 234-244, wherein said pharmaceutical composition comprises semaglutide and one or more excipients and has a pH in the range of about 5.6 to about 9.0.
246. The pharmaceutical composition according to embodiment 245, wherein said pharmaceutical composition has a pH of about 7.4.
247. The pharmaceutical composition according to any one of embodiments 234-246, wherein said method further comprises subcutaneous administration of about 0.25-16 mg cagrilintide per week, such as once weekly.
248. The pharmaceutical composition according to any one of embodiments 234-247, wherein said cagrilintide is administered in an amount per week, such as once weekly, of about 0.25 to about 9.0 mg, about 0.25 to about 4.5 mg or about 0.25 to about 2.4 mg.
249. The pharmaceutical composition according to embodiment 248, wherein said cagrilintide is administered in an amount per week, such as once weekly, of about 2.4 mg or about 3.6 mg.
250. The pharmaceutical composition according to any one of embodiments 234-249, for use in a method for treating or reducing the risk of one or more selected from the group consisting of
   (i) cardiovascular (CV) disease, such as reducing the risk of major adverse cardiovascular event comprising CV death, non-fatal myocardial infarction and non-fatal stroke,
   (ii) metabolic dysfunction-associated steatohepatitis, and
   (iii) chronic kidney disease in a human subject in need thereof,
wherein the method comprises subcutaneous administration of about 7.2 mg semaglutide per week.
251. Pharmaceutical composition, characterized by comprising semaglutide or a pharmaceutically acceptable salt thereof.
252. Pharmaceutical composition according to claim 251, characterized in that the concentration of semaglutide is in the range of 0.1 mg/ml to 100 mg/ml.
253. Pharmaceutical composition according to claim 251 or 252, characterized by further comprising one or more additional active ingredients.
254. Pharmaceutical composition according to any one of claims 251 to 253, characterized in that it is for subcutaneous administration.
255. Pharmaceutical composition according to any one of claims 251 to 254, characterized in that it is a subcutaneous injectable solution.
256. Pharmaceutical composition according to any one of claims 251 to 255, characterized in that it has a pH in the range of 5.6 to 10.0.
257. Pharmaceutical composition according to any one of claims 251 to 256, characterized in that it is free of cyclodextrin.
258. Pharmaceutical composition, characterized by comprising semaglutide or a pharmaceutically acceptable salt thereof, to be used in the form of a subcutaneous injectable aqueous solution.
259. Pharmaceutical composition according to any one of claims 251 to 258, characterized in that it is contained in a medical device, such as a vial or injection device.
260. Pharmaceutical composition according to claim 259, characterized in that said medical device contains 251 to 254 single doses of up to 7.2 mg of semaglutide or a pharmaceutically acceptable salt thereof.
261. Pharmaceutical composition according to claim 260, characterized in that the single doses are weekly doses.
262. Pharmaceutical composition according to any one of claims 259 to 261, characterized in that the medical device is capable of releasing up to 3 subsequent doses of 2.4 mg of semaglutide or a pharmaceutically acceptable salt thereof per day.
263. Pharmaceutical composition according to claim 259, characterized in that the medical device is capable of releasing a single amount of up to 7.2 mg of semaglutide or a pharmaceutically acceptable salt thereof.
264. Pharmaceutical composition according to any one of claims 259 to 263, characterized in that the medical device contains a total of up to 28.8 mg of semaglutide or a pharmaceutically acceptable salt thereof.
265. Pharmaceutical composition, according to any one of claims 251 to 264, characterized in that it is for weight management of a human subject.
266. Pharmaceutical composition according to any one of claims 251 to 264, characterized in that it is for treating, preventing and/or reducing the risk of a disease.
267. Pharmaceutical composition, according to claim 266, characterized in that the disease is selected from the group consisting of:
   (i) obesity;
   (ii) overweight;
   (iii) cardiovascular (CV) disease, such as reducing the risk of major adverse cardiovascular event comprising CV death, non-fatal myocardial infarction and non-fatal stroke;
   (iv) metabolic dysfunction-associated steatohepatitis;
   (v) chronic kidney disease;
   (vi) type 2 diabetes; and
   (vii) Alzheimer's disease.
268. Kit, characterized by comprising a pharmaceutical composition as defined in any one of claims 251 to 267, and instructions for use.
269. Kit, characterized by comprising semaglutide, or a pharmaceutically acceptable salt thereof, and instructions for use.
270. Kit, characterized by comprising semaglutide, or a pharmaceutically acceptable salt thereof, and instructions for administering semaglutide in an amount of up to 7.2 mg once a week to a subject in need thereof.
271. Kit, characterized by comprising:
   (i) a medical device containing a pharmaceutical composition which comprises 1 to 4 single doses of up to 7.2 mg of semaglutide, or a pharmaceutically acceptable salt thereof, and
   (ii) instructions for administering semaglutide in an amount of up to 7.2 mg once a week to a subject in need thereof.
272. Kit, characterized by comprising:
   (i) a medical device containing a pharmaceutical composition which comprises 1 to 4 single doses of 2.4 mg semaglutide, or a pharmaceutically acceptable salt thereof, and
   (ii) instructions for weekly administering 3 doses at once to a subject in need thereof.
273. Kit, characterized by comprising:
   (i) at least one medical device containing a pharmaceutical composition comprising semaglutide; and
   (ii) instructions for use.
274. Kit according to claim 273, characterized in that said medical device contains:
   (a) 1 to 4 single doses of up to 7.2 mg semaglutide or a pharmaceutically acceptable salt thereof; or
   (b) a total of up to 28.8 mg of semaglutide or a pharmaceutically acceptable salt thereof.
275. Kit, characterized by comprising:
   (i) a first medical device, wherein said first medical device contains 1 to 4 single doses of up to 7.2 mg semaglutide, or a pharmaceutically acceptable salt thereof;
   (ii) a second medical device, wherein said medical device contains 1 to 4 single doses of up to 16 mg of an amylin receptor agonist or a pharmaceutically acceptable salt thereof; and
   (iii) instructions for use.
276. Medical device, characterized in that it contains:
   (a) 1 to 4 single doses of up to 7.2 mg of semaglutide or a pharmaceutically acceptable salt thereof; or
   (b) a total of up to 28.8 mg of semaglutide or a pharmaceutically acceptable salt thereof.
277. Medical device, characterized by comprising semaglutide in a first chamber and an amylin receptor agonist in a second chamber,
   wherein the first chamber contains 1 to 4 single doses of up to 7.2 mg semaglutide or a pharmaceutically acceptable salt thereof, and
   wherein the second chamber contains 1 to 4 single doses of up to 16 mg of an amylin receptor agonist or a pharmaceutically acceptable salt thereof.
278. Use of semaglutide or a pharmaceutically acceptable salt thereof, characterized by being for the manufacture of a medicament, pharmaceutical composition, combination and/or medical device, for treating, preventing and/or reducing the risk of a disease.
279. Use of semaglutide or a pharmaceutically acceptable salt thereof, characterized by being for the manufacture of a medicament, pharmaceutical composition, combination and/or medical device, in which the concentration of semaglutide is in the range of 0.1 mg/ml to 100 mg/ml, for treating, preventing and/or reducing the risk of a disease.
280. Use of semaglutide or a pharmaceutically acceptable salt thereof, characterized by being for the manufacture of a pharmaceutical composition in which the concentration of semaglutide is in the range of 0.1 mg/ml to 100 mg/ml, for treating, preventing and/or reducing the risk of a disease, in which the composition contains 1 to 4 single doses of up to 7.2 mg of semaglutide or a pharmaceutically acceptable salt thereof.
281. Use of semaglutide or a pharmaceutically acceptable salt thereof, characterized by being for the manufacture of a pharmaceutical composition in which the concentration of semaglutide is in the range of 0.1 mg/ml to 100 mg/ml, for treating, preventing and/or reducing the risk of a disease,
   where the composition is contained in a medical device,
   wherein the medical device contains 1 to 4 single doses of up to 7.2 mg semaglutide or a pharmaceutically acceptable salt thereof.
282. Use of semaglutide or a pharmaceutically acceptable salt thereof, characterized by being for the manufacture of a medicament, pharmaceutical composition, combination and/or medical device, in combination with an amylin receptor agonist or a pharmaceutically acceptable salt thereof, for treating, preventing and/or reducing the risk of a disease.
283. Use of semaglutide or a pharmaceutically acceptable salt thereof, and an amylin receptor agonist or a pharmaceutically acceptable salt thereof, characterized by being for the manufacture of a medicament, pharmaceutical composition, combination and/or medical device, for treating, preventing and/or reducing the risk of a disease.
284. Use according to any one of claims 278 to 283, characterized in that the disease is selected from the group consisting of:
   (i) obesity;
   (ii) overweight;
   (iii) cardiovascular (CV) disease, such as reducing the risk of major adverse cardiovascular event comprising CV death, non-fatal myocardial infarction and non-fatal stroke;
   (iv) metabolic dysfunction-associated steatohepatitis;
   (v) chronic kidney disease;
   (vi) type 2 diabetes; and
   (vii) Alzheimer's disease.

### Non-Limiting Aspects of the Invention

The invention is further described by the following non-limiting aspects:
1. Use of a unit dosage form of semaglutide in the manufacture of a pharmaceutical composition for the weight management of a subject in need thereof, wherein the unit dosage form of semaglutide comprises up to about 7.2 mg semaglutide.
2. Use of a unit dosage form of semaglutide in the manufacture of a pharmaceutical composition for the weight management of a subject in need thereof, wherein the unit dosage form of semaglutide comprises about 6.0 mg to about 7.2 mg semaglutide.
3. Use of a unit dosage form of semaglutide in the manufacture of a pharmaceutical composition for the weight management of a subject in need thereof, wherein the unit dosage form of semaglutide comprises about 7.2 mg semaglutide.
4. Use of a unit dosage form of semaglutide in the manufacture of a pharmaceutical composition for the treatment of obesity or overweight in a subject in need thereof, wherein the unit dosage form of semaglutide comprises about 7.2 mg semaglutide.
5. Use of a unit dosage form of semaglutide in the manufacture of a pharmaceutical composition for the treatment of obesity or overweight in a subject in need thereof, wherein said subject further has at least one weight-related co-morbidity, wherein the unit dosage form of semaglutide comprises about 7.2 mg semaglutide.
6. The use according to aspect 5, wherein said weight-related co-morbidity is selected from the group consisting of diabetes, cardiovascular disease, metabolic dysfunction-associated steatohepatitis, alcoholic liver disease, and obstructive sleep apnoea.
7. The use according to any one of the preceding aspects, wherein the unit dosage form is a single weekly dose.
8. The use according to any one of the preceding aspects, wherein said pharmaceutical composition comprises 1 to 4 unit dosage forms.
9. The use according to any one of the preceding aspects, wherein said pharmaceutical composition comprises 1 or 4 unit dosage forms.
10. The use according to any one of the preceding aspects, wherein said pharmaceutical composition comprises 1 unit dosage form.
11. The use according to any one of the preceding aspects, wherein said pharmaceutical composition is a solution.
12. The use according to any one of the preceding aspects, wherein said pharmaceutical composition is an aqueous solution.
13. The use according to any one of the preceding aspects, wherein said pharmaceutical composition further comprises an amylin receptor agonist, preferably, the amylin receptor agonist is cagrilintide.
14. The use according to any one of the preceding aspects, wherein said pharmaceutical composition is contained in a medical device, e.g. an injection device or a vial, preferably, the injection device is a pen-injector.
15. The use according to any one of the preceding aspects, wherein the unit dosage form comprises three subunit dosage forms, e.g. each subunit dosage form of semaglutide comprises 2.4 mg semaglutide.
16. The use according to aspect 15, wherein the subunit dosage form of semaglutide is contained in a medical device, e.g. an injection device or a vial, preferably, the injection device is a pen-injector.
17. The use according to aspect 14, wherein the medical device comprises semaglutide in a first chamber and an amylin receptor agonist in a second chamber.
18. The use according to aspect 17, wherein the second chamber contains 1 to 4 single weekly doses of up to 16 mg of an amylin receptor agonist or a pharmaceutically acceptable salt thereof, preferably, the amylin receptor agonist is cagrilintide.
19. The use according to any one of the preceding aspects, wherein said subject has a BMI of at least 28 kg/m2.
20. The use according to any one of the preceding aspects, wherein said subject has a BMI of at least 30 kg/m2.
21. The use according to any one of the preceding aspects, wherein said subject has a BMI of at least 35 kg/m2.
22. The use according to any one of the preceding aspects, wherein said subject has a BMI of at least 40 kg/m2.
23. The use according to any one of the preceding aspects, wherein said subject has a BMI of 28-50 kg/m2.
24. The use according to any one of the preceding aspects, wherein said subject has a BMI of 28-45 kg/m2.
25. The use according to any one of the preceding aspects, wherein said subject has a BMI of 28-40 kg/m2.
26. The use according to any one of the preceding aspects, wherein said subject has a BMI of 28-35 kg/m2.
27. The use according to any one of the preceding aspects, wherein said subject has a BMI of at least 24 kg/m2.
28. The use according to any one of the preceding aspects, wherein said subject has a BMI of 24-28 kg/m2.
29. The use according to any one of the preceding aspects, wherein said subject has a BMI of least 28 kg/m2 and a body weight of at least about 100 kg.
30. The use according to any one of the preceding aspects, wherein said subject has a BMI of least 28 kg/m2 and a body weight of at least about 120 kg.
31. The use according to any one of the preceding aspects, wherein said subject has a

BMI of least 28 kg/m2 and a body weight of at least about 140 kg.
32. The use according to any one of the preceding aspects, wherein said subject is adult or paediatric.
33. The use according to any one of the preceding aspects, wherein said subject is paediatric aged 12 years or older.
34. The use according to any one of the preceding aspects, wherein said subject has diabetes.
35. The use according to any one of the preceding aspects, wherein said subject has diabetes, such as type 2 diabetes.
36. The use according to any one of the preceding aspects, wherein said subject has at least one weight-related comorbid condition, such as dyslipidemia or hypertension.
37. The use according to any one of the preceding aspects, wherein said subject has overweight and at least one weight-related comorbid condition, such as dyslipidemia or hypertension.
38. The use according to any one of the preceding aspects, wherein said subject has a BMI of at least 24 kg/m2 and at least one weight-related comorbid condition, such as dyslipidemia or hypertension.
39. The use according to any one of the preceding aspects, wherein said subject is not classified as New York Heart Association (NYHA) class IV.
40. The use according to any one of the preceding aspects, wherein said pharmaceutical composition is for subcutaneous administration.
41. The use according to any one of the preceding aspects, wherein said pharmaceutical composition is administered per week.
42. The use according to any one of the preceding aspects, wherein said pharmaceutical composition is administered once weekly.
43. The use according to any one of the preceding aspects, wherein said pharmaceutical composition is administered for at least 24 weeks.
44. The use according to any one of the preceding aspects, wherein said pharmaceutical composition is administered for at least 32 weeks.
45. The use according to any one of the preceding aspects, wherein said pharmaceutical composition is administered for at least 40 weeks.
46. The use according to any one of the preceding aspects, wherein said pharmaceutical composition is administered for at least 52 weeks.
47. The use according to any one of the preceding aspects, wherein said pharmaceutical composition is administered for at least 72 weeks.
48. The use according to any one of the preceding aspects, wherein said pharmaceutical composition is administered for about 72 weeks.
49. The use according to any one of the preceding aspects, wherein said pharmaceutical composition is administered for at least about 72 weeks from the initial semaglutide dosage escalation step.
50. The use according to any one of the preceding aspects, wherein said pharmaceutical composition is administered for up to about 72 weeks from the initial semaglutide dosage escalation step.
51. The use according to any one of the preceding aspects, wherein said pharmaceutical composition is administered for at least about 72 weeks including dosage escalation.
52. The use according to any one of the preceding aspects, wherein said pharmaceutical composition is administered for about 72 weeks including dosage escalation.
53. The use according to any one of the preceding aspects, wherein said weight management or treatment provides a body weight reduction of said subject.
54. The use according to any one of the preceding aspects, wherein said body weight reduction is at least about 15%, such as at least 18% or at least 20%.
55. The use according to any one of the preceding aspects, wherein said body weight reduction is at least about 15%.
56. The use according to any one of the preceding aspects, wherein said body weight reduction is at least 18%.
57. The use according to any one of the preceding aspects, wherein said body weight reduction is at least 20%.
58. The use according to any one of the preceding aspects, wherein said body weight reduction is at least 25%.
59. The use according to any one of the preceding aspects, wherein said weight management or treatment provides a reduction of lean body volume compared to total fat volume of less than 20%, such as less than 18% or less than 16%.
60. The use according to any one of the preceding aspects, wherein said weight management or treatment provides a reduction of lean body volume compared to total fat volume of no more than 15%.
61. The use according to any one of the preceding aspects, wherein said weight management or treatment provides a reduction of lean body volume compared to total fat volume of about 10% to about 20%.
62. The use according to any one of the preceding aspects, wherein said weight management or treatment is tolerable for said subject.
63. The use according to any one of the preceding aspects, wherein less than about 15% of subjects discontinue administration of said pharmaceutical composition.
64. The use according to any one of the preceding aspects, wherein about 8% to about 15% of subjects discontinue administration of said pharmaceutical composition.
65. The use according to any one of the preceding aspects, wherein about 8% to about 15% of subjects discontinue administration of said pharmaceutical composition.
66. The use according to any one of the preceding aspects, wherein less than about 15% of subjects discontinue administration of said pharmaceutical composition within at least about 52 weeks.
67. The use according to any one of the preceding aspects, wherein less than 15% of subjects discontinue administration of said pharmaceutical composition within at least about 52 weeks after an initial dosage escalation, wherein said dosage escalation is as defined herein.
68. The use according to any one of the preceding aspects, wherein less than 12% of subjects discontinue administration of said pharmaceutical composition.
69. The use according to any one of the preceding aspects, wherein less than 12% of subjects discontinue administration of said pharmaceutical composition within at least about 52 weeks.
70. The use according to any one of the preceding aspects, wherein less than 12% of subjects discontinue administration of said pharmaceutical composition within at least about 52 weeks after an initial dosage escalation, wherein said dosage escalation is as defined herein.
71. The use according to any one of the preceding aspects, wherein said discontinue is a permanent discontinuation, such as a discontinuation for at least 4 weeks or at least 8 weeks.
72. The use according to any one of the preceding aspects, wherein less than about 15% of subjects discontinue administration of said pharmaceutical composition and said weight management or treatment provides a body weight reduction of at least about 15%.
73. The use according to any one of the preceding aspects, wherein less than about 15% of subjects discontinue administration of said pharmaceutical composition and said weight management or treatment provides a body weight reduction of at least about 15%.
74. The use according to any one of the preceding aspects, wherein less than about 15% of subjects discontinue administration of said pharmaceutical composition and said weight management or treatment provides a body weight reduction of at least about 15%.
75. The use according to any one of the preceding aspects, wherein about 65% to about 75% of subjects have at least one gastrointestinal adverse event.
76. The use according to any one of the preceding aspects, wherein about 65% to about 75% of subjects have at least one gastrointestinal adverse event during administration of said pharmaceutical composition within about 52 weeks.
77. The use according to any one of the preceding aspects, wherein about 71% % of subjects have at least one gastrointestinal adverse event during administration of said pharmaceutical composition within about 52 weeks.
78. The use according to any one of the preceding aspects, wherein about 71% % of subjects have at least one gastrointestinal adverse event during administration of said pharmaceutical composition within about 72 weeks including dosage escalation.
79. The use according to any one of the preceding aspects, wherein about 65% to about 75% of subjects have at least one gastrointestinal adverse event.
80. The use according to any one of the preceding aspects, wherein about 35% to about 50% of subjects have at least one gastrointestinal adverse event.
81. The use according to any one of the preceding aspects, wherein about 40% to about 48% of subjects have at least one gastrointestinal adverse event.
82. The use according to any one of the preceding aspects, wherein about 44% of subjects have at least one gastrointestinal adverse event.
83. The use according to any one of the preceding aspects, wherein the waist circumference of said subject is reduced.
84. The use according to any one of the preceding aspects, wherein the blood pressure of said subject is reduced.
85. The use according to any one of the preceding aspects, wherein the diastolic blood pressure and/or systolic blood pressure of said subject is reduced.
86. The use according to any one of the preceding aspects, wherein the diastolic blood pressure and systolic blood pressure of said subject is reduced.
87. The use according to any one of the preceding aspects, wherein said semaglutide is ad-ministered via subcutaneous injection.
88. The use according to any one of the preceding aspects, wherein said pharmaceutical com-position comprises semaglutide and one or more excipients.
89. The use according to any one of the preceding aspects, wherein said pharmaceutical com-position consists essentially of semaglutide and one or more excipients.
90. The use according to any one of the preceding aspects, wherein said pharmaceutical com-position consists of semaglutide and one or more excipients.
91. The use according to any one of the preceding aspects, wherein said pharmaceutical com-position consists of semaglutide and one or more excipients and said composition has a pH in the range of about 5.5 to about 10.0.
92. The use according to any one of the preceding aspects, wherein said pharmaceutical com-position has a pH in the range of about 5.6 to about 10.0.
93. The use according to any one of the preceding aspects, wherein said pharmaceutical com-position has a pH in the range of about 6.0 to about 9.0.
94. The use according to any one of the preceding aspects, wherein said pharmaceutical com-position has a pH in the range of about 6.8 to about 8.0.
95. The use according to any one of the preceding aspects, wherein said pharmaceutical com-position has a pH in the range of about 7.0 to about 7.8.
96. The use according to any one of the preceding aspects, wherein said pharmaceutical com-position has a pH in the range of about 7.2 to about 7.6.
97. The use according to any one of the preceding aspects, wherein said pharmaceutical com-position has a pH of about 7.4.
98. The use according to any one of the preceding aspects, wherein said pharmaceutical com-position comprises a buffer.
99. The use according to any one of the preceding aspects, wherein said pharmaceutical com-position comprises a phosphate buffer.
100. The use according to any one of the preceding aspects, wherein said pharmaceutical composition comprises an isotonic agent.
101. The use according to any one of the preceding aspects, wherein said pharmaceutical composition comprises sodium chloride.
102. The use according to any one of the preceding aspects, wherein said pharmaceutical composition does not comprise cyclodextrin.
103. The use according to any one of the preceding aspects, wherein said pharmaceutical composition does not comprise a preservative.
104. The use according to any one of the preceding aspects, wherein said pharmaceutical composition comprises a preservative.
105. The use according to any one of the preceding aspects, wherein said pharmaceutical composition is administered via an injection device.
106. Use of about 7.2 mg semaglutide in the manufacture of a pharmaceutical composition for treating or reducing the risk of cardiovascular disease in a subject in need thereof.
107. The use according to the preceding aspect, wherein said use is as defined in any one of aspects 7-105.
108. Use of about 7.2 mg semaglutide in the manufacture of a pharmaceutical composition for reducing the risk of major adverse cardiovascular event comprising CV death, non-fatal myocardial infarction and non-fatal stroke in a subject in need thereof.
109. The use according to the preceding aspect, wherein said use is as defined in any one of aspects 7-105.
110. Use of about 7.2 mg semaglutide in the manufacture of a pharmaceutical composition for treating or reducing the risk of heart failure in a subject in need thereof.
111. The use according to the preceding aspect, wherein said use is as defined in any one of aspects 7-105.
112. Use of about 7.2 mg semaglutide in the manufacture of a pharmaceutical composition for treating metabolic dysfunction-associated steatohepatitis (MASH) in a subject in need thereof.
113. The use according to the preceding aspect, wherein said use is as defined in any one of aspects 7-105.
114. Use of about 7.2 mg semaglutide in the manufacture of a pharmaceutical composition for treatment of alcoholic liver disease (ALD) in a subject in need thereof.
115. The use according to the preceding aspect, wherein said use is as defined in any one of aspects 7-105.
116. Use of about 7.2 mg semaglutide in the manufacture of a pharmaceutical composition for treatment of type 2 diabetes in a subject in need thereof.
117. The use according to the preceding aspect, wherein said use is as defined in any one of aspects 7-105.
118. Use of about 7.2 mg semaglutide in the manufacture of a pharmaceutical composition for treatment of Alzheimer's disease in a subject in need thereof.
119. The use according to the preceding aspect, wherein said use is as defined in any one of aspects 7-105.
120. Use of about 7.2 mg semaglutide in the manufacture of a pharmaceutical composition for treatment of chronic kidney disease in a subject in need thereof.
121. The use according to the preceding aspect, wherein said use is as defined in any one of aspects 7-105.
122. Use of about 7.2 mg semaglutide in the manufacture of a pharmaceutical composition for treatment of peripheral arterial disease in a subject in need thereof.
123. The use according to the preceding aspect, wherein said use is as defined in any one of aspects 7-105.
124. Use of about 7.2 mg semaglutide in the manufacture of a pharmaceutical composition for treatment of obstructive sleep apnoea in a subject in need thereof.
125. The use according to the preceding aspect, wherein said use is as defined in any one of aspects 7-105.
126. The use according to any one of the preceding aspects, wherein said Alzheimer's disease is early Alzheimer's disease.
127. The use according to any one of the preceding aspects, wherein said heart failure is heart failure with preserved ejection fraction.
128. The use according to any one of the preceding aspects, wherein said treatment comprises administration of a further active ingredient.
129. The use according to any one of the preceding aspects, wherein said treatment further comprises administration of cagrilintide.
130. The use according to any one of the preceding aspects, wherein said cagrilintide is administered simultaneously with or sequentially to said semaglutide.
131. The use according to any one of the preceding aspects, wherein said cagrilintide is administered via subcutaneous administration.
132. The use according to any one of the preceding aspects, wherein said cagrilintide is administered via subcutaneous injection.
133. The use according to any one of the preceding aspects, wherein said cagrilintide is about 0.25-16 mg.
134. The use according to any one of the preceding aspects, wherein said cagrilintide is about 0.25-9.0 mg.
135. The use according to any one of the preceding aspects, wherein said cagrilintide is about 0.25-4.5 mg.
136. The use according to any one of the preceding aspects, wherein said cagrilintide is about 0.25 mg.
137. The use according to any one of the preceding aspects, wherein said cagrilintide is about 0.5 mg.
138. The use according to any one of the preceding aspects, wherein said cagrilintide is about 1.0 mg.
139. The use according to any one of the preceding aspects, wherein said cagrilintide is about 1.7 mg.
140. The use according to any one of the preceding aspects, wherein said cagrilintide is about 2.4 mg.
141. The use according to any one of the preceding aspects, wherein said cagrilintide is about 3.6 mg.
142. The use according to any one of the preceding aspects, wherein said cagrilintide is administered once weekly.
143. The use according to any one of the preceding aspects, wherein said cagrilintide is administered per week.
144. The use according to any one of the preceding aspects, wherein said pharmaceutical composition comprises cagrilintide and one or more excipients.
145. The use according to any one of the preceding aspects, wherein said pharmaceutical composition comprises cagrilintide, semaglutide and one or more excipients.
146. The use according to any one of the preceding aspects, wherein said pharmaceutical composition consists of cagrilintide, semaglutide and one or more excipients.
147. The use according to any one of the preceding aspects, wherein said pharmaceutical composition comprising cagrilintide is a solution.
148. The use according to any one of the preceding aspects, wherein said pharmaceutical composition comprising cagrilintide is an aqueous solution.
149. The use according to any one of the preceding aspects, wherein the pharmaceutical composition comprising cagrilintide and semaglutide has a pH in the range of 5.6-6.0.
150. The use according to any one of the preceding aspects, wherein the pharmaceutical composition comprising cagrilintide and semaglutide has a pH in the range of 5.8-6.2.
151. The use according to any one of the preceding aspects, wherein said pharmaceutical composition comprising cagrilintide has a pH of about 3.5-4.5, such as pH 4.0.
152. The use according to any one of the preceding aspects, wherein said pharmaceutical composition comprising cagrilintide comprises a buffer.
153. The use according to any one of the preceding aspects, wherein said pharmaceutical composition comprising cagrilintide comprises a glutamic acid, glutamate, lactic acid, lactate, acetic acid, or acetate buffer.
154. The use according to any one of the preceding aspects, wherein said pharmaceutical composition comprising cagrilintide comprises a glutamic acid or glutamate buffer.
155. The use according to any one of the preceding aspects, wherein said pharmaceutical composition comprising cagrilintide comprises a lactic acid or lactate buffer.
156. The use according to any one of the preceding aspects, wherein said pharmaceutical composition comprising cagrilintide comprises an acetic acid or acetate buffer.
157. A pharmaceutical combination, comprising 1) a first dose comprising about 0.25 mg semaglutide; and/or 2) a second dose comprising about 0.5 mg semaglutide; 3) and/or a third dose comprising about 1.0 mg semaglutide; 4) and/or a fourth dose comprising about 1.7 mg semaglutide; 5) and/or a fifth dose comprising about 2.4 mg semaglutide; and 6) a sixth dose comprising about 7.2 mg semaglutide.
158. The pharmaceutical combination according to aspect 157, wherein each dose is a single weekly dose.
159. The pharmaceutical combination according to aspect 157, wherein each dose is from single dose form or multiple dose form.
160. The pharmaceutical combination according to aspect 159, wherein the single dose form comprises one weekly dose.
161. The pharmaceutical combination according to aspect 159, wherein the multiple dose form comprises 2-4 weekly doses, such as 4 weekly doses.
162. The pharmaceutical combination according to aspect 157, wherein the sixth dose is administered with 3 unit dosage forms of 2.4 mg semaglutide.
163. The pharmaceutical combination according to aspect 157, wherein the dosage escalation for each dose from 0.25 mg to 7.2 mg is performed every four weeks.

### EXAMPLES

### Example 1: Effect and Safety of Semaglutide 7.2 mg Once-Weekly in Patients with Obesity

An interventional, multi-national, multi-centre, randomised, parallel group, double-blind, placebo-controlled, three-armed 72-week clinical trial with semaglutide was performed in 1407 adult human patients with obesity. Patients received once-weekly subcutaneous administration of either semaglutide 7.2 mg, semaglutide 2.4 mg or placebo (herein also simply referred to as “semaglutide 7.2 mg”, “semaglutide 2.4 mg”, and “placebo”, respectively), as an adjunct to lifestyle intervention (reduced-calorie diet and increased physical activity), from start to end of the clinical trial (week 0 to week 72).

For patients administered semaglutide, dosage escalations were performed every four weeks: 0.25 mg (week 0), 0.5 mg (week 4), 1.0 mg (week 8), 1.7 mg (week 12), and 2.4 mg (week 16); as well as 2.4 mg (week 20) or 7.2 mg (week 20) for Semaglutide 2.4 mg and Semaglutide 7.2 mg, respectively. Weeks 21-72 were a 52-week maintenance period with once-weekly subcutaneous administration of either semaglutide 7.2 mg, semaglutide 2.4 mg or placebo. Weeks 73-82 were a 9-week follow-up period during which semaglutide 7.2 mg, semaglutide 2.4 mg or placebo was not administered. Lifestyle intervention also ended at week 72. Table 1 lists details of inclusion and exclusion criteria for patients in the clinical trial. “Screening” occurred shortly before the first administration of semaglutide or placebo, such as about one week prior to said first administration. A sub-population of the randomised patients had their body composition assessed by MRI (magnetic resonance imaging) at the beginning and at the end of the clinical trial to investigate the degree to which weight loss was caused by reduction in fat mass. For the MRI subpopulation, MRI was performed from vertebrae T9 to the knees. Baseline characteristics of all randomised patients are listed in Table 2a and for the MRI subpopulation in Table 2b. The term “baseline” as used herein refers to start of the clinical trial (week 0) and before the first administration of semaglutide or placebo.

Semaglutide was administered in the form of an aqueous solution at pH of about 7.4. The aqueous solution comprising semaglutide may further comprise 1.42 mg/ml disodium phosphate, dihydrate and 8.25 mg/ml sodium chloride. The term “placebo” as used herein refers to a formulation identical to the semaglutide formulation except not comprising semaglutide. Semaglutide 2.4 mg and placebo were administered in the volume used in the equivalent semaglutide 7.2 mg dosage, also during dosage escalation.

The primary endpoints were (i) change in body weight (%) from baseline (week 0) to end of treatment (week 72) and (ii) proportion of patients with body weight reduction≥5% at end of treatment (week 72). Secondary endpoints included proportion of patients with body weight reduction≥15%, ≥20%, or ≥25% at end of treatment.

TABLE 1
Inclusion and exclusion criteria

| | |
|---|---|
| Inclusion | Patients were eligible to be included in the clinical trial only if all the following |
| criteria | criteria applied: (i) Male or female; (ii) Age above or equal to 18 years; (iii) |
| Exclusion | Body mass index (BMI)^{a} ≥ 30.0 kg/m²; and (iv) History of at least one self- |
| criteria | reported unsuccessful dietary effort to lose body weight. |
| | For the MRI sub-population, all of the following further inclusion criteria |
| | applied: (i) Body weight < 149.0 kg; (ii) No metal implants e.g., pacemaker, |
| | intracranial clips, cochlear implant, infusion pump or neurostimulators; and |
| | (iii) Suitable for MRI as per Investigator's discretion e.g., no claustrophobia. |
| | Patients were excluded from the clinical trial if any of the following criteria |
| | applied: (i) Known or suspected hypersensitivity to study intervention(s) or |
| | related products; (ii) Female who is pregnant, breast-feeding or intends to |
| | become pregnant or is of childbearing potential and not using highly effective |
| | contraceptive method; (iii) Participation (i.e., signed informed consent) in any |
| | interventional, clinical trial within 90 days before screening; (iv) HbA1c ≥ 48 |
| | mmol/mol (6.5%); (v) History of type 1 or type 2 diabetes; (vi) Treatment with |
| | glucose-lowering agent(s) within 90 days before screening; (vii) Treatment |
| | with GLP-1 receptor agonist within 180 days prior to screening; (viii) A self- |
| | reported change in body weight > 5 kg (11 lbs) within 90 days before |
| | screening irrespective of medical records; (ix) Treatment with any medication |
| | for the indication of obesity within 90 days before screening; (x) Previous or |
| | planned (during the trial period) obesity treatment with surgery or a weight |
| | loss device. However, the following are allowed: 1) liposuction and/or |
| | abdominoplasty, if performed >1 year before screening, 2) lap banding, if the |
| | band has been removed >1 year before screening, 3) intragastric balloon, if |
| | the balloon has been removed >1 year before screening, or 4) duodenal- |
| | jejunal bypass sleeve, if the sleeve has been removed >1 year before |
| | screening; (xi) Uncontrolled thyroid disease per investigator's discretion; (xii) |
| | Presence of acute pancreatitis within 180 days before screening; (xiii) History |
| | or presence of chronic pancreatitis; (xiv) Personal or first-degree relative(s) |
| | history of multiple endocrine neoplasia type 2 or medullary thyroid carcinoma; |
| | (xv) Renal impairment measured as estimated Glomerular Filtration Rate |
| | (eGFR) value of eGFR <15 mL/min/1.73 m² as defined by KDIGO 2012 |
| | (Kidney Int Suppl. 2013; 3(1): 1-150) at screening; (xvi) Presence or history of |
| | malignant neoplasm (other than basal or squamous cell skin cancer, in-situ |
| | carcinomas of the cervix, or in situ prostate cancer) within 5 years prior to |
| | screening; (xvii) Any of the following: myocardial infarction, stroke, |
| | hospitalisation for unstable angina or transient ischaemic attack within 60 |
| | days before screening; (xviii) Patients presently classified as being in New |
| | York Heart Association (NYHA) class IV; (xiv) Surgery scheduled for the |
| | duration of the trial, expect for minor surgical procedures, in the opinion of the |
| | investigator; (xv) Known or suspected abuse of alcohol or recreational drugs; |
| | (xvi) Other patient(s) from the same household participating in any |
| | semaglutide clinical trial; (xvii) Any disorder, unwillingness or inability, not |
| | covered by any of the other exclusion criteria, which in the investigator's |
| | opinion, might jeopardise the patient's safety or compliance with the protocol; |
| | (xviii) Active or unstable depression or other active or unstable psychiatric |
| | conditions which in the investigator's opinion can jeopardize patient's safety |
| | or compliance with the clinical trial protocol; (xix) A Patient Health |
| | Questionaire-9 (PHQ-9) score of ≥15 at screening; (xx) A lifetime history of |
| | suicidal attempt; (xxi) Suicidal behaviour within 30 days before screening; |
| | (xxii) Suicidal ideation corresponding to type 4 or 5 on the Columbia-Suicide |
| | Severity Rating Scale (C-SSRS) within 30 days before screening. |

Baseline characteristics of patients are shown in Table 2a and 2b for all randomised patients and the MRI subpopulation, respectively.

TABLE 2a
Baseline characteristics of all randomised patients

| | Semaglutide | Semaglutide | |
|---|---|---|---|
| | 7.2 mg | 2.4 mg | Placebo |
| N | 1005 | 201 | 201 |
| Age ^{a} | 47 (12) | 46 (12) | 48 (12) |
| Body weight ^{a} [kg] | 112.4 (23.8) | 116.5 (26.2) | 112.4 (22.8) |
| BMI ^{a} [kg/m²] | 39.8 (7.0) | 40.5 (7.8) | 39.7 (6.6) |
| Waist circumference ^{a} [cm] | 118.4 (15.8) | 120.3 (16.9) | 118.6 (14.5) |
| HbA1c ^{a} [%] | 5.7 (0.3) | 5.6 (0.4) | 5.7 (0.3) |
| eGFR ^{b} [mL/min/1.73 m²] | 95.1 (19.6) | 96.4 (18.0) | 95.5 (20.4) |

N: Number of patients.
^{a} mean (SD).
SD: Standard deviation.
^{b} geometric mean (CV).
CV: Coefficient of variation.

TABLE 2b
Baseline characteristics of the MRI subpopulation

| | Semaglutide | Semaglutide | Semaglutide, | |
|---|---|---|---|---|
| | 7.2 mg | 2.4 mg | pooled | Placebo |
| N | 43 | 6 | 49 | 6 |
| Age ^{a} | 44 (11) | 49 (15) | 45 (12) | 43 (9) |
| Body weight ^{a} [kg] | 108.4 (18.8) | 120.7 (21.6) | 109.9 (19.3) | 117.9 (23.1) |
| BMI ^{a} [kg/m²] | 39.1 (5.7) | 38.0 (6.5) | 39.0 (5.8) | 42.1 (6.9) |
| Waist circumference ^{a} [cm] | 119.0 (12.6) | 123.2 (15.9) | 119.5 (12.9) | 121.8 (14.6) |
| HbA1c ^{a} [%] | 5.6 (0.3) | 5.5 (0.4) | 5.6 (0.3) | 5.6 (0.2) |
| eGFR ^{b} [mL/min/1.73 m²] | 101.7 (16.2) | 99.8 (20.6) | 101.4 (16.6) | 88.8 (35.6) |

N: Number of patients.
^{a} mean (SD).
SD: Standard deviation.
^{b} geometric mean (CV).
CV: Coefficient of variation.

Results may be presented as either on-treatment, for all randomised subjects or hypothetical estimand. The term “on-treatment” as used herein refers to all data points where patients are treated with trial product (semaglutide or placebo), i.e. excluding any off-treatment time intervals triggered by at least two consecutive missed dosages. The term “hypothetical estimand” as used herein refers to statistical analyses of data based on the assumption that all patients remained on their trial product (semaglutide or placebo) for the entire planned duration of the clinical trial and not initiated any other anti-obesity therapy (weight management drugs or bariatric surgery); this estimation employed a mixed model for repeated measurements (MMRM) using on-treatment data until first discontinuation of trial product or initiation of other anti-obesity therapies; the MMRM was fitted with randomised treatment as factor and baseline value as covariate all nested within visit as a factor; missing week 72 values were predicted from the MMRM.

Results are shown in Tables 3-10.

TABLE 3
Change from baseline at week 72 for the hypothetical
estimand. Results presented as estimated mean (SE),
where SE is the standard error of the mean.

| | Semaglutide | Semaglutide | |
|---|---|---|---|
| | 7.2 mg | 2.4 mg | Placebo |
| Body weight reduction (%) | −20.7 (0.33) | −17.5 (0.75) | −2.4 (0.76) |
| Waist circumference | −18.9 (0.34) | −15.9 (0.75) | −4.9 (0.78) |
| HbA1c (%) | −0.4 (0.01) | −0.3 (0.02) | 0.0 (0.02) |
| HbA1c (mmol/mol) | −4.0 (0.09) | −3.5 (0.21) | 0.5 (0.22) |
| Body Weight (Kg) | −22.9 (0.38) | −19.2 (0.84) | −2.8 (0.86) |
| BMI (kg/m²) | −8.1 (0.13) | −6.8 (0.30) | −1.0 (0.31) |
| Fasting plasma glucose (mg/dL) | −12.5 (0.32) | −11.0 (0.71) | −1.2 (0.75) |
| Systolic blood pressure (mmHg) | −10.8 (0.41) | −9.4 (0.94) | −4.1 (0.99) |
| Diastolic blood pressure (mmHg) | −4.9 (0.28) | −3.5 (0.63) | −2.1 (0.67) |
| Waist Height Ratio | −0.1 (0.00) | −0.1 (0.00) | −0.0 (0.00) |

The results in Table 3 show that semaglutide 7.2 mg provided significantly greater body weight reduction compared to semaglutide 2.4 mg or placebo, that semaglutide 7.2 mg provided significantly greater blood pressure reduction, both for HbA1c and fasting plasma glucose (FPG), compared to placebo, and that semaglutide 7.2 mg provided significantly greater blood pressure reduction, both for systolic blood pressure and diastolic blood pressure, compared to placebo.

TABLE 4
Body weight reduction compared to baseline at 72
weeks for all randomised patients on-treatment

| Body weight | Semaglutide 7.2 mg | | Semaglutide 2.4 mg | | Placebo | |
|---|---|---|---|---|---|---|
| reduction | N | % | N | % | N | % |
| ≥5% | 814 | 93.2 | 161 | 92.5 | 50 | 35.7 |
| ≥10% | 751 | 86.0 | 135 | 77.6 | 28 | 20.0 |
| ≥15% | 615 | 70.4 | 100 | 57.5 | 11 | 7.9 |
| ≥20% | 444 | 50.9 | 61 | 35.1 | 4 | 2.9 |
| ≥25% | 290 | 33.2 | 29 | 16.7 | 0 | 0 |

N: Number of patients.
%: Proportion of patients in each group.

The results in Table 4 show that for semaglutide 7.2 mg more patients obtained a body weight reduction of ≥5%, ≥10%, ≥15%, ≥20%, or ≥25% compared to semaglutide 2.4 mg or placebo. In addition, significantly more patients obtained a body weight reduction of ≥15%, ≥20%, or ≥25% compared to semaglutide 2.4 mg or placebo.

TABLE 5
BMI Category-shift-in-trial-at week 72

| | BMI category at week 0 | | | | |
|---|---|---|---|---|---|
| Treatment | Normal | Over- | Obesity | Obesity | Obesity |
| Category | weight | weight | class I | class II | class III |
| (at week 72) | N (%) | N (%) | N (%) | N (%) | N (%) |

Sema 7.2 mg

| | | | | | |
|---|---|---|---|---|---|
| Normal weight | 0 | 3 (75.0) | 95 (36.4) | 41 (14.0) | 9 (2.3) |
| Overweight | 0 | 1 (25.0) | 124 (47.5) | 107 (36.5) | 30 (7.7) |
| Obesity class I | 0 | 0 | 40 (15.3) | 106 (36.2) | 104 (26.5) |
| Obesity class II | 0 | 0 | 2 (0.8) | 36 (12.3) | 131 (33.4) |
| Obesity class III | 0 | 0 | 0 | 3 (1.0) | 118 (30.1) |

Sema 2.4 mg

| | | | | | |
|---|---|---|---|---|---|
| Normal weight | 0 | 0 | 10 (21.3) | 3 (5.3) | 1 (1.2) |
| Overweight | 0 | 0 | 27 (57.4) | 19 (33.3) | 7 (8.3) |
| Obesity class I | 0 | 0 | 9 (19.1) | 27 (47.4) | 14 (16.7) |
| Obesity class II | 0 | 0 | 1 (2.1) | 8 (14.0) | 27 (32.1) |
| Obesity class III | 0 | 0 | 0 | 0 | 35 (41.7) |

Placebo

| | | | | | |
|---|---|---|---|---|---|
| Normal weight | 0 | 0 | 0 | 0 | 0 |
| Overweight | 0 | 0 | 13 (28.9) | 1 (1.9) | 0 |
| Obesity class I | 0 | 0 | 27 (60.0) | 19 (35.2) | 5 (6.9) |
| Obesity class II | 0 | 0 | 5 (11.1) | 32 (59.3) | 10 (13.9) |
| Obesity class III | 0 | 0 | 0 | 2 (3.7) | 57 (79.2) |

N: Number of subjects,
%: Percentages in relation to subjects with respective treatment and BMI category at week 0

The results in Table 5 show that in semaglutide 7.2 mg more subjects had a shift to lower BMI categories at week 72 when compared to baseline than in semaglutide 2.4 mg or placebo.

TABLE 6
BMI <27 kg/m² obtained at week 72 for all randomised
patients on-treatment. Results are presented as N (%),
where N is the number of patients and % is the
proportion of patients in each group.

| Semaglutide 7.2 mg | Semaglutide 2.4 mg | Placebo |
|---|---|---|
| 245 (28.1) | 35 (20.1) | 0 (0) |

The results in Table 6 show that a significantly higher proportion of patients obtained a BMI of less than 27 kg/m² for semaglutide 7.2 mg compared to semaglutide 2.4 mg or placebo.

TABLE 7
Blood pressure change from baseline (mmHg) at 72 weeks
for all randomised patients on-treatment. Results are
presented as mean (SD), where SD is standard deviation.

| | Semaglutide | Semaglutide | |
|---|---|---|---|
| | 7.2 mg | 2.4 mg | Placebo |
| Systolic blood pressure | −11 (13) | −8 (14) | −4 (13) |
| Diastolic blood pressure | −5 (9) | −3 (9) | −1 (10) |

The results in Table 7 surprisingly show that semaglutide 7.2 mg provided a greater blood pressure reduction, both for systolic blood pressure and diastolic blood pressure, compared to semaglutide 2.4 mg or placebo.

TABLE 7a
Body composition change from baseline at 72 weeks for
all randomised patients in the MRI subpopulation. Results are
presented as mean (liter) and SD for semaglutide 7.2 mg
and estimated mean and SE for semaglutide pooled.

| | Semaglutide | Semaglutide |
|---|---|---|
| | 7.2 mg^{#} | pooled * |
| Total fat volume | −14 (8.2) | −12.7 (0.97) |
| Visceral fat volume | −2.0 (1.2) | −1.9 (0.17) |
| Lean body volume | −2.3 (1.8) | −2.3 (0.30) |

* All semaglutide dosages pooled. Analysis of data from on-treatment period using MMRM.
^{#}Mean of observed data.

The results in Table 7a surprisingly show that only 16.4% lean body volume compared to total fat volume (2.3/14.0) was lost with 7.2 mg semaglutide for the MRI subpopulation (semaglutide 7.2 mg) whereas 18.1% lean body volume compared to total fat volume (2.3/12.7) was lost with pooled semaglutide for the MRI subpopulation (semaglutide pooled).

TABLE 7b
Body composition change from baseline at 72 weeks for all
randomised patients in the MRI subpopulation. Results are
presented as % relative to baseline MRI parameters.

| | Semaglutide | Semaglutide |
|---|---|---|
| | 7.2 mg | pooled * |
| Total fat volume | −32.1 (16.5) | −29.7 |
| Visceral fat volume | −39.7 (20.6) | −36.5 |
| Lean body volume | −9.5 (6.6) | −9.6 |

* All semaglutide dosages pooled. Analysis of data from on-treatment period using MMRM.
#: Mean of observed data.

TABLE 7c
Waist-to-height ratio (WHtR) of <0.50 and waist circumference
change from baseline at 72 weeks for all randomised
patients on-treatment. WHtR is calculated as waist
circumference divided by height. Results are presented as mean.

| | Semaglutide | Semaglutide | |
|---|---|---|---|
| | 7.2 mg | 2.4 mg | Placebo |
| Waist-to-height ratio <0.50 | 142 (16.3) | 13 (7.5) | 2 (1.4) |
| Waist circumference (cm) | −18.9 (0.34) | −15.9 (0.75) | −4.9 (0.78) |

The results in Table 7c surprisingly show that for semaglutide 7.2 mg a greater proportion of patients achieved a waist-to-height ratio (WHIR) of less than 0.50, in addition to a greater waist circumference (WC) reduction, compared to semaglutide 2.4 mg or placebo. At week 72, a greater proportion of patients treated with semaglutide 7.2 mg (14.6%) achieved WHIR<0.50, compared with 2.4 mg (6.5%) and placebo (1.1%). At week 72, patients in the semaglutide 7.2 mg, semaglutide 2.4 mg and placebo groups had mean reductions in WC of −18.9 cm, −15.9 cm and −4.9 cm, respectively. The estimated treatment ratio (ETR) [95% CI] for semaglutide 7.2 mg vs semaglutide 2.4 mg was 2.5 [95% CI 1.5, 4.1]; p=0.0014 for waist-to-height ratio <0.50 and the estimated treatment difference (ETD) [95% CI] for semaglutide 7.2 mg vs semaglutide 2.4 mg was −3.0 [95% Cl −4.6, −1.4; p=0.0003) for waist circumference.

TABLE 8
Discontinuations at week 72 based on all randomised patients.
Results are presented as N (%), where N is the number of
patients and % is the proportion of patients in each group.

| Semaglutide 7.2 mg | Semaglutide 2.4 mg | Placebo |
|---|---|---|
| 117 (11.6) | 24 (11.9) | 59 (29.4) |

The results in Table 8 surprisingly show that tolerability of semaglutide 7.2 mg was better than expected since for semaglutide 7.2 mg the proportion of patients discontinuing the clinical trial was similar to that for semaglutide 2.4 mg (11.6% and 11.9%, respectively); also, the proportion of patients discontinuing the clinical trial was greater for placebo than for semaglutide 7.2 mg or semaglutide 2.4 mg. This is particularly surprising in view of the dosage escalation directly from 2.4 mg to 7.2 mg semaglutide.

TABLE 9
Gastrointestinal adverse event (GI-AE), nausea, diarrhea, vomiting
or constipation reported from week 0-72 for all randomised
patients on-treatment. Results are presented as N (%), where N is
the number of patients experiencing at least one event and % is the
proportion of patients in each group experiencing at least one event.

| | Semaglutide | Semaglutide | |
|---|---|---|---|
| | 7.2 mg | 2.4 mg | Placebo |
| GI-AE * | 711 (70.8) | 123 (61.2) | 86 (42.8) |
| Nausea | 439 (43.7) | 77 (38.3) | 26 (12.9) |
| Diarrhea | 272 (27.1) | 56 (27.9) | 26 (12.9) |
| Vomiting | 249 (24.8) | 33 (16.4) | 14 (7.0) |
| Constipation | 233 (23.2) | 39 (19.4) | 18 (9.0) |

* Including nausea, diarrhoea, vomiting, and constipation.

The results in Table 9 show the proportion of gastrointestinal adverse events, including nausea, diarrhea, vomiting, and constipation, for semaglutide 7.2 mg, semaglutide 2.4 mg and placebo. Surprisingly, tolerability of semaglutide 7.2 mg was better than expected since for semaglutide 7.2 mg the proportion of patients with diarrhea was similar to that for semaglutide 2.4 mg (27.1% and 27.9%, respectively).

TABLE 10a
Proportion of patients experiencing at least one of the adverse
events at any time while on-treatment: (i) gastrointestinal
adverse event (GI-AE), (ii) nausea or (iii) vomiting, from
week 0-72 within exposure level-based subgroups
characterized by their median semaglutide plasma
concentration level (nmol/liter). Results for GI-AE, nausea
or vomiting are presented as mean (%). Semaglutide plasma
concentration is presented as the median (nmol/liter).

| Semaglutide | | | |
|---|---|---|---|
| (nmol/liter) | GI-AE | Nausea | Vomiting |
| 0.0 | 42.8 | 12.9 | 7.0 |
| 82.3 | 64.6 | 39.6 | 16.2 |
| 191 | 65.0 | 37.9 | 20.0 |
| 226 | 67.5 | 37.5 | 21.7 |
| 259 | 78.3 | 50.8 | 30.8 |
| 303 | 71.2 | 48.3 | 28.3 |

TABLE 10b
Semaglutide plasma concentration for patients
on-treatment. Semaglutide plasma concentration
is presented as the median (nmol/liter).

| Treatment group | Semaglutide (nmol/liter) |
|---|---|
| Semaglutide 2.4 mg | 76.4 |
| Semaglutide 7.2 mg | 238 |

The results in Table 10a show an increase of the adverse events of gastrointestinal adverse events, nausea and vomiting with increasing semaglutide plasma concentration.

The results in Tables 10a-10b are based on semaglutide plasma concentration value at steady state for each subject determined from a population pharmacokinetics (PK) model and based on the randomized treatment group dosage level. For patients administered semaglutide, results in Table 10a were obtained based on (a) these semaglutide plasma concentration values at steady state for each subject by sorting patients administered semaglutide from lowest to highest semaglutide plasma concentration, dividing this sorted list of patients into five equally sized subgroups of around 250 patients, and then determining the median semaglutide plasma concentration was determined for each subgroup and (b) the mean proportion of patients experiencing at least one adverse event for each of GI-AE, nausea or vomiting was determined for each subgroup. For patients administered placebo, results in Table 10a were obtained based on (a) a semaglutide plasma concentration of 0 and (b) the mean proportion of patients experiencing at least one adverse event for each of GI-AE, nausea or vomiting was determined for the placebo group.

The results in Table 10b show that the median semaglutide plasma concentration was 238 nmol/liter for semaglutide 7.2 mg and 76.4 in nmol/liter for semaglutide 2.4 mg.

TABLE 10c
Ratio to baseline at week 72 for the hypothetical estimand.

| | | Sema- | Sema- | |
|---|---|---|---|---|
| | | glutide | glutide | |
| | | 7.2 mg | 2.4 mg | Placebo |
| | | Est. | Est. | Est. |
| | Parameters | Ratio | Ratio | Ratio |
| 1 | Fasting serum insulin (pmol/L) | 0.58 | 0.67 | 0.94 |
| 2 | Fasting serum insulin (mIU/mL) | 0.58 | 0.67 | 0.94 |
| 3 | Total cholesterol (mmol/L) | 0.94 | 0.94 | 1.00 |
| 4 | Total cholesterol (mg/dL) | 0.94 | 0.94 | 1.00 |
| 5 | HDL cholesterol (mmol/L) | 1.08 | 1.05 | 1.02 |
| 6 | HDL cholesterol (mg/dL) | 1.08 | 1.05 | 1.02 |
| 7 | LDL cholesterol (mmol/L) | 0.92 | 0.92 | 0.97 |
| 8 | LDL cholesterol (mg/dL) | 0.92 | 0.92 | 0.97 |
| 9 | VLDL cholesterol (mmol/L) | 0.74 | 0.83 | 0.99 |
| 10 | VLDL cholesterol (mg/dL) | 0.74 | 0.83 | 0.99 |
| 11 | Triglycerides (mmol/L) | 0.74 | 0.83 | 0.99 |
| 12 | Triglycerides (mg/dl) | 0.74 | 0.83 | 0.99 |
| 13 | hsCRP (mg/L) | 0.37 | 0.41 | 0.89 |

The fasting serum insulin results in Table 10c (Rows 1-2) show that semaglutide 7.2 mg provided significantly greater reduction of fasting serum insulin compared to placebo.

The lipid results in Table 11 (Rows 3-13) show that semaglutide 7.2 mg provided significantly greater reduction of Total cholesterol, LDL cholesterol, VLDL cholesterol, Triglycerides and hsCRP, and provided significantly greater increase of HDL cholesterol compared to placebo.

TABLE 10d
Glycaemic Category - shift from Pre-diabetes at baseline
to Normo-glycaemia at week 72 -in-trial.

| | Semaglutide | Semaglutide | |
|---|---|---|---|
| Parameters | 7.2 mg | 2.4 mg | Placebo |
| Glycaemic shift | 297 (83.4) | 48 (73.8) | 26 (36.6) |

N: Number of participants.
%: Percentages are based on participants with an observation at the visit.

The results in Table 10d shows that in semaglutide 7.2 mg more subjects had a change in their glycaemic status from pre-diabetes to normoglycaemia when compared to semaglutide 2.4 mg or placebo.

### Example 2: Effect and Safety of Semaglutide 7.2 mg Once-Weekly in Participants with Obesity and Type 2 Diabetes

An interventional, multi-national, multi-centre, randomised, parallel group, double-blind, placebo-controlled, three-armed 72-week clinical trial with semaglutide was performed in 512 adult human patients with obesity and type 2 diabetes. Patients received once-weekly subcutaneous administration of either semaglutide 7.2 mg, semaglutide 2.4 mg or placebo (herein also simply referred to as “semaglutide 7.2 mg”, “semaglutide 2.4 mg”, and “placebo”, respectively), as an adjunct to lifestyle intervention (reduced-calorie diet and increased physical activity), from start to end of the clinical trial (week 0 to week 72).

For patients administered semaglutide, dosage escalations were performed every four weeks: 0.25 mg (week 0), 0.5 mg (week 4), 1.0 mg (week 8), 1.7 mg (week 12), and 2.4 mg (week 16); as well as 2.4 mg (week 20) or 7.2 mg (week 20) for Semaglutide 2.4 mg and Semaglutide 7.2 mg, respectively. Weeks 21-72 were a 52-week maintenance period with once-weekly subcutaneous administration of either semaglutide 7.2 mg, semaglutide 2.4 mg or placebo. Weeks 73-82 were a 9-week follow-up period during which semaglutide 7.2 mg, semaglutide 2.4 mg or placebo was not administered. Lifestyle intervention also ended at week 72. Table 11 lists details of inclusion and exclusion criteria for patients in the clinical trial. “Screening” occurred shortly before the first administration of semaglutide or placebo, such as about one week prior to said first administration. Baseline characteristics of all randomised patients are listed in Table 12. The term “baseline” as used herein refers to start of the clinical trial (week 0) and before the first administration of semaglutide or placebo.

Semaglutide was administered in the form of an aqueous solution at pH of about 7.4. The aqueous solution comprising semaglutide may further comprise 1.42 mg/ml disodium phosphate, dihydrate and 8.25 mg/ml sodium chloride. The term “placebo” as used herein refers to a formulation identical to the semaglutide formulation except not comprising semaglutide. Semaglutide 2.4 mg and placebo were administered in the volume used in the equivalent semaglutide 7.2 mg dosage, also during dosage escalation.

The primary endpoints were (i) change in body weight (%) from baseline (week 0) to end of treatment (week 72) and (ii) proportion of patients with body weight reduction≥5% at end of treatment (week 72). Secondary endpoints included proportion of patients with body weight reduction≥20% at end of treatment.

TABLE 11

| | Inclusion and exclusion criteria |
|---|---|
| Inclusion | Patients were eligible to be included in the clinical trial only if all the following |
| criteria | criteria applied: (i) Male or female; (ii) Age above or equal to 18 years; (iii) |
| | Body mass index (BMI) ≥ 30.0 kg/m²; (iv) History of at least one self-reported |
| | unsuccessful dietary effort to lose body weight; (v) diagnosed with type 2 |
| | diabetes ≥180 days prior to the day of screening; (vi) HbA1c 7.0-10.0% (53- |
| | 86 mmol/mol) at screening; and (vii) treatment with either (a) 1-3 marketed |
| | oral antidiabetic drugs (metformin, glinide, thiazolidinedione, SGLT2i, α- |
| | glucosidase inhibitors, or sulfonylureas) and same oral anti-diabetic drug(s), |
| | dose and dosing frequency required for at least 90 days before screening or |
| | (b) lifestyle intervention. |
| Exclusion | Patients were excluded from the clinical trial if any of the following criteria |
| criteria | applied: (i) Known or suspected hypersensitivity to study intervention(s) or |
| | related products; (ii) Female who is pregnant, breast-feeding or intends to |
| | become pregnant or is of childbearing potential and not using highly effective |
| | contraceptive method; (iii) Participation (i.e., signed informed consent) in any |
| | interventional, clinical trial within 90 days before screening; (iv) Receipt of |
| | any other anti-diabetic investigational drug within 90 days prior to screening, |
| | or receipt of any investigational drugs not affecting diabetes within 30 days |
| | prior to screening; (v) Treatment with GLP-1 receptor agonist within 180 days |
| | prior to screening; (vi) Renal impairment with estimated Glomerular Filtration |
| | Rate (eGFR) < 30 mL/min/1.73 m² (<45 mL/min/1.73 m² in participants |
| | treated with SGLT2i) according to CKD-EPI creatinine equation as defined by |
| | KDIGO 2012 (Kidney Int Suppl. 2013; 3(1): 1-150) at screening; (vii) |
| | Uncontrolled and potentially unstable diabetic retinopathy or maculopathy as |
| | verified by a fundus examination performed within 90 days before screening |
| | or in the period between screening and randomisation (pharmacological |
| | pupil-dilation is a requirement unless using a digital fundus photography |
| | camera specified for non-dilated examination); (viii) A self-reported change in |
| | body weight > 5 kg (11 lbs) within 90 days before screening irrespective of |
| | medical records; (ix) Treatment with any medication prescribed for the |
| | indication of obesity or weight management within 90 days before screening; |
| | (x) Previous or planned (during the clinical trial period) obesity treatment with |
| | surgery or a weight loss device. However, the following are allowed: 1) |
| | liposuction and/or abdominoplasty, if performed >1 year before screening, 2) |
| | lap banding, if the band has been removed >1 year before screening, 3) |
| | intragastric balloon, if the balloon has been removed >1 year before |
| | screening, or 4) duodenal-jejunal bypass sleeve, if the sleeve has been |
| | removed >1 year before screening; (xi) Uncontrolled thyroid disease as per |
| | investigator's discretion; (xii) Active or unstable depression or other active or |
| | unstable psychiatric conditions which in the investigator's opinion can |
| | jeopardize participant's safety or compliance with the clinical trial protocol; |
| | (xiii) A Patient Health Questionnaire-9 (PHQ-9) score of ≥15 at screening; |
| | (xiv) A lifetime history of suicidal attempt; (xv) Suicidal behaviour within 30 |
| | days before screening; (xvi) Suicidal ideation corresponding to type 4 or 5 on |
| | the Columbia-Suicide Severity Rating Scale (C-SSRS) within 30 days before |
| | screening; (xvii) Presence of acute pancreatitis within 180 days before |
| | screening; (xviii) History or presence of chronic pancreatitis; (xix) Personal or |
| | first-degree relative(s) history of multiple endocrine neoplasia type 2 or |
| | medullary thyroid carcinoma; (xx) Presence or history of malignant neoplasm |
| | (other than basal or squamous cell skin cancer, in-situ carcinomas of the |
| | cervix, or in situ prostate cancer) within 5 years prior to screening; (xxi) Any |
| | of the following: myocardial infarction, stroke, hospitalisation for unstable |
| | angina or transient ischaemic attack within 60 days before screening; (xxii) |
| | Participants presently classified as being in New York Heart Association |
| | (NYHA) class IV; (xxiii) Surgery scheduled for the duration of the study, |
| | expect for minor surgical procedures, in the opinion of the investigator; (xxiv) |
| | Known or suspected abuse of alcohol or recreational drugs; (xxv) Any |
| | disorder, unwillingness or inability, not covered by any of the other exclusion |
| | criteria, which in the investigator's opinion, might jeopardise the participant's |
| | safety or compliance with the protocol. |

Baseline characteristics of patients are shown in Table 12 for all randomised patients.

TABLE 12
Baseline characteristics of all randomised patients

| | Semaglutide | Semaglutide | |
|---|---|---|---|
| | 7.2 mg | 2.4 mg | Placebo |
| N | 307 | 103 | 102 |
| Age ^{a} | 57 (10) | 58 (10) | 55 (10) |
| Body weight ^{a} [kg] | 110.5 (22.9) | 107.0 (23.0) | 112.1 (22.9) |
| BMI ^{a} [kg/m²] | 38.7 (7.1) | 37.7 (6.4) | 39.0 (7.6) |
| Waist circumference ^{a} [cm] | 121.8 (15.2) | 119.1 (13.2) | 123.9 (15.5) |
| HbA1c ^{a} [%] | 8.0 (0.8) | 8.1 (0.9) | 8.2 (0.9) |
| eGFR ^{b} [mL/min/1.73m²] | 92.5 (21.3) | 91.5 (22.3) | 92.6 (25.6) |

N: Number of patients.
^{a} mean (SD).
SD: Standard deviation.
^{b} geometric mean (CV).
CV: Coefficient of variation.

Results may be presented as either on-treatment, for all randomised subjects or hypothetical estimand. The term “on-treatment” as used herein refers to all data points where patients are treated with trial product (semaglutide or placebo), i.e. excluding any off-treatment time intervals triggered by at least two consecutive missed dosages. The term “hypothetical estimand” as used herein refers to statistical analyses of data based on the assumption that all patients remained on their trial product (semaglutide or placebo) for the entire planned duration of the clinical trial and not initiated any other anti-obesity therapy (weight management drugs or bariatric surgery); this estimation employed a mixed model for repeated measurements (MMRM) using on-treatment data until first discontinuation of trial product or initiation of other anti-obesity therapies or initiation of anti-diabetic rescue medication (only applicable to objectives addressing glucose metabolism); the MMRM was fitted with randomised treatment as factor and baseline value as covariate all nested within visit as a factor; missing week 72 values were predicted from the MMRM.

Results are shown in Tables 13-17.

TABLE 13
Change from baseline at week 72 for the hypothetical estimand.
Results presented as estimated mean and standard error.

| | Semaglutide | Semaglutide | |
|---|---|---|---|
| | 7.2 mg | 2.4 mg | Placebo |
| Body weight reduction (%) | −14.1 (0.49) | −10.7 (0.84) | −3.6 (0.84) |
| Waist circumference | −13.5 (0.56) | −11.1 (0.97) | −6.0 (0.97) |
| HbA1c (%) | −1.9 (0.065) | −1.6 (0.111) | 0.1 (0.124) |
| HbA1c (mmol/mol) | −20.5 (0.712) | −17.6 (1.214) | 1.1 (1.36) |
| Body Weight (Kg) | −15.6 (0.56) | −11.8 (0.96) | −4.1 (0.96) |
| BMI (kg/m²) | −5.5 (0.20) | −4.2 (0.34) | −1.4 (0.34) |
| Fasting plasma glucose | −63.3 (2.62) | −54.5 (4.45) | −7.6 (5.31) |
| (mg/dL) | | | |
| Systolic blood pressure | −8.2 (0.74) | −6.61 (1.25) | −2.97 (1.28) |
| (mmHg) | | | |
| Waist Height Ratio | −0.1 (0) | −0.1 (0.01) | −0.0 (0.01) |

The results in Table 13 show that semaglutide 7.2 mg provided greater body weight reduction compared to semaglutide 2.4 mg or placebo. The results in Table 13 also show that semaglutide 7.2 mg provided greater reduction in waist circumference, HbA1c, body weight, BMI, fasting plasma glucose, and systolic blood pressure compared to semaglutide 2.4 mg and placebo.

TABLE 14
Body weight reduction compared to baseline at 72
weeks for all randomised patients on-treatment

| Body weight | Semaglutide 7.2 mg | | Semaglutide 2.4 mg | | Placebo | |
|---|---|---|---|---|---|---|
| reduction | N | % | N | % | N | % |
| ≥5% | 243 | 89.7 | 72 | 78.3 | 28 | 33.3 |
| ≥10% | 178 | 65.7 | 50 | 54.3 | 11 | 13.1 |
| ≥15% | 117 | 43.2 | 28 | 30.4 | 7 | 8.3 |
| ≥20% | 61 | 22.5 | 14 | 15.2 | 2 | 2.4 |

N: Number of patients.
%: Proportion of patients in each group.

The results in Table 14 show that for semaglutide 7.2 mg more patients obtained a body weight reduction of ≥5%, ≥10%, ≥15%, or ≥20% compared to semaglutide 2.4 mg or placebo.

TABLE 15
Discontinuations at week 72 based on all randomised patients.
Results are presented as N (%), where N is the number of
patients and % is the proportion of patients in each group.

| Semaglutide 7.2 mg | Semaglutide 2.4 mg | Placebo |
|---|---|---|
| 29 (9.4) | 9 (8.7) | 17 (16.7) |

The results in Table 15 surprisingly show that tolerability of semaglutide 7.2 mg was better than expected since for semaglutide 7.2 mg the proportion of patients discontinuing the clinical trial was similar to that for semaglutide 2.4 mg (9.4% and 8.7%, respectively); also, the proportion of patients discontinuing the clinical trial was greater for placebo than for semaglutide 7.2 mg or semaglutide 2.4 mg. This is particularly surprising in view of the dosage escalation directly from 2.4 mg to 7.2 mg semaglutide.

TABLE 16
Gastrointestinal adverse event (GI-AE), nausea, diarrhea,
vomiting or constipation reported from week 0-72 for
all randomised patients on-treatment. Results are
presented as N (%), where N is the number of patients
experiencing at least one event and % is the proportion
of patients in each group experiencing at least one event.

| | Semaglutide | Semaglutide | |
|---|---|---|---|
| | 7.2 mg | 2.4 mg | Placebo |
| GI-AE * | 163 (53.1) | 53 (51.5) | 26 (25.5) |
| Nausea | 89 (29.0) | 29 (28.2) | 12 (11.8) |
| Diarrhea | 56 (18.2) | 21 (20.4) | 9 (8.8) |
| Vomiting | 51 (16.6) | 14 (13.6) | 3 (2.9) |
| Constipation | 45 (14.7) | 19 (18.4) | 5 (4.9) |

* Including nausea, diarrhoea, vomiting, and constipation.

The results in Table 16 show gastrointestinal adverse events, including nausea, diarrhea, vomiting and constipation, for semaglutide 7.2 mg, semaglutide 2.4 mg, and placebo. Surprisingly, the results are similar for semaglutide 7.2 mg and semaglutide 2.4 mg, in particular a smaller portion of patients (%) experienced diarrhea and constipation for semaglutide 7.2 mg compared to semaglutide 2.4 mg.

TABLE 17
Ratio to baseline at week 72 for the hypothetical estimand.

| | Semaglutide | Semaglutide | Placebo |
|---|---|---|---|
| | 7.2 mg | 2.4 mg | Est. |
| Parameters | Est. Ratio | Est. Ratio | Ratio |
| HDL cholesterol (mmol/L) | 1.11 | 1.05 | 1.06 |
| HDL cholesterol (mg/dL) | 1.11 | 1.05 | 1.06 |
| VLDL cholesterol (mmol/L) | 0.71 | 0.79 | 0.90 |
| VLDL cholesterol (mg/dl) | 0.71 | 0.79 | 0.90 |
| Free fatty acids (mmol/L) | 0.73 | 0.79 | 1.01 |
| Free fatty acids (mg/dl) | 0.73 | 0.79 | 1.01 |
| Triglycerides (mmol/L) | 0.70 | 0.77 | 0.89 |
| Triglycerides (mg/dL) | 0.70 | 0.77 | 0.89 |
| hsCRP (mg/L) | 0.44 | 0.46 | 0.80 |

### Example 3: Co-Administration of Semaglutide and Cagrilintide in Pigs

Studies were conducted in LYD pigs to evaluate the pharmacokinetic (PK) profile of combining cagrilintide and semaglutide administration either (1) via a single s.c. administration with prefilled dual chamber syringe or (2) via separate injections of each drug. The volumes administered were about 500 microlitres of a semaglutide formulation and about 250 microlitres of a cagrilintide formulation. Dose ranges of from 0.25 mg to 2.4 mg semaglutide and from 0.25 to 4.5 mg cagrilintide were explored.

Female cross bred domestic pigs (Danish Landrace, Yorkshire and Duroc-LYD pigs), having a body weight of approximately 80-100 kg, were used for exploring PK profiles. The low dose was dosed subcutaneously with a single dose of semaglutide/cagrilintide as monotherapy or in combination using either a prefilled dual chamber syringe or as separate injections at different injections sites using the NovoPen 4 penfill (Novo Nordisk A/S). Before dosing the high doses of semaglutide/cagrilintide, the LYD pigs were dose-escalated with liraglutide, for 10 days, to avoid an initial gastro-intestinal tract related discomfort from an acute high dose of semaglutide. On day 11, the animals were dosed with semaglutide/cagrilintide as described above.

A full plasma concentration-time profile was obtained from each animal, from pre-dosing to 18 days post dosing.

Plasma was analysed by means of Luminescence Oxygen Channelling Immunoassay (LOCI) and Liquid Chromatography-Mass Spectrometry (LCMS) for semaglutide and cagrilintide, respectively. Non-compartmental pharmacokinetic analysis, using individual plasma concentration-time profiles, was performed.

TABLE 18
Comparison of PK parameters of semaglutide after co-dosing of semaglutide/cagrilintide 2.4 mg/4.5
mg using separate injections versus using a prefilled dual chamber syringe (mean ± SD)

| | Semaglutide | Cagrilintide | Semaglutide | Cagrilintide | | |
|---|---|---|---|---|---|---|
| | Cmax | Cmax | AUC/Dose | AUC/Dose | Semaglutide | Cagrilintide |
| | (nmol/L) | (nmol/L) | (hr*kg/L) | (hr*kg/L) | t½ (hr) | t½ (hr) |
| Group | [mean ± SD] | [mean ± SD] | [mean ± SD] | [mean ± SD] | [mean ± SD] | [mean ± SD] |
| Mono | 124 ± 23.6 | 42.0 ± 4.54 | 1380 ± 78.1 | 1160 ± 75.5 | 46.5 ± 4.38 | 87.8 ± 6.93 |
| Dual | 151 ± 33.4 | 69.3 ± 11.8 | 1560 ± 229 | 1690 ± 300 | 46.5 ± 4.14 | 88.3 ± 6.38 |
| chamber | | | | | | |
| syringe | | | | | | |
| 2.4/2.4 | | | | | | |
| % increase | ~22% | ~65% | ~13% | ~45% | ~0% | ~0.6% |
| (Co-dosing | | | | | | |
| vs. dual- | | | | | | |
| chamber | | | | | | |
| syringe) | | | | | | |

A moderate difference in semaglutide PK parameters, Cₘₐₓ and tₘₐₓ, was observed with the different administration methods. For cagrilintide Cₘₐₓ was increased up to 65% and AUC increased up to 45% following administration of 2.4/2.4 semaglutide/cagrilintide with a prefilled dual chamber syringe, as opposed to when administered with semaglutide via two different devices.

These non-clinical data indicates an improved effect when semaglutide and cagrilintide are administered in a single injection using a dual-chamber syringe; as opposed to when they are dosed separately, using individual syringes such as the NovoPen 4 penfill.

### Example 4: Co-Administration of Semaglutide and Cagrilintide in a Phase 1 Human Trial

### Trial Design

A 20-week, multiple ascending dose phase 1 trial investigated the safety, tolerability, pharmacokinetics and weight loss potential of AM833 (cagrilintide) administered in combination with 2.4 mg semaglutide. In the trial, six different cagrilintide doses (0.16 mg, 0.3 mg, 0.6 mg, 1.2 mg, 2.4 mg and 4.5 mg per week) were administered with semaglutide (2.4 mg per week), as separate subcutaneous injections to six separate cohorts. Subjects were randomised 3:1 to receive cagrilintide+2.4 mg semaglutide or placebo+2.4 mg semaglutide. A 16-week dose-escalation period was applied followed by 4 weeks at the target dose. 80 adults with an initial BMI between 27.0 and 39.9 kg/m² (both inclusive) completed the trial.

The trial investigated the number of treatment-emergent adverse events (primary endpoint). AM833 was well-tolerated, with the most common adverse events being injection site reactions and gastrointestinal disorders, including nausea and vomiting, the majority being non-serious and mild or moderate in severity. Surprisingly, the level of gastrointestinal disorders observed for the combination of AM833 and semaglutide in the trial was comparable to what is generally seen for glucagon-like peptides-1 (GLP-1) in monotherapy.

### Results

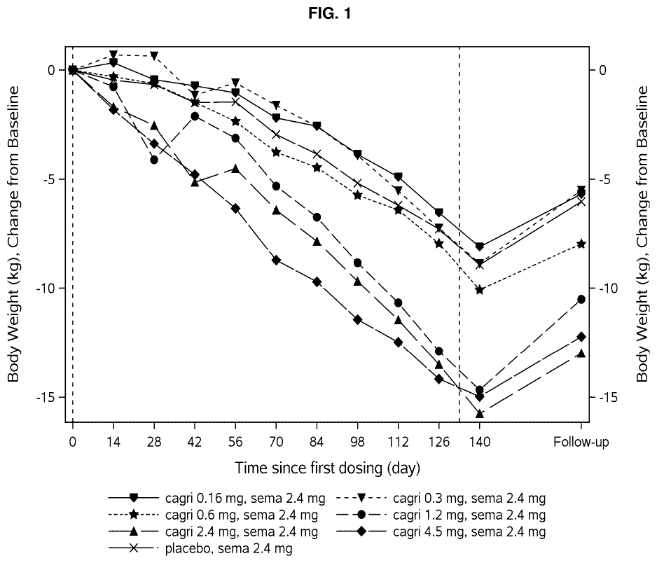

From a mean baseline body weight of 95.7 kg, body weight decreased in all treatment arms over the 20-week treatment period, with substantial weight loss observed in subjects receiving the three highest doses of cagrilintide (1.2 mg, 2.4 mg and 4.5 mg)+2.4 mg semaglutide compared to placebo+2.4 mg semaglutide (FIG. 1).

After 20 weeks of treatment, there was a statistically significant treatment difference for mean change from baseline to end of treatment in body weight for the three highest doses of cagrilintide (1.2 mg, 2.4 mg and 4.5 mg) in combination with semaglutide, in comparison to placebo+semaglutide. The estimated mean body weight change from baseline to end of treatment were 15.6% for cagrilintide 1.2 mg, 17.0% for cagrilintide 2.4 mg and 15.6% for cagrilintide 4.5 mg; all in combination with 2.4 mg of semaglutide. A weight loss of 9.8% was observed for placebo+semaglutide. Treatment differences were not statistically significant for the three lowest doses of cagrilintide (0.16 mg, 0.3 mg and 0.6 mg) in combination with semaglutide, when compared to placebo in combination with 2.4 mg semaglutide. In all treatment arms there was evidence of body weight regain following cessation of the use of trial products at Week 20.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1.-41. (canceled)

42. A method for weight management of a human subject in need thereof, comprising subcutaneously administering semaglutide in an amount of about 7.2 mg weekly to the human subject, wherein semaglutide is the only active ingredient administered for weight management.

43. The method according to claim 42, wherein the human subject has obesity.

44. The method according to claim 42, wherein the human subject has overweight.

45. The method according to claim 42, wherein prior to the administering of the about 7.2 mg per week of semaglutide, administering semaglutide to the subject in one or more dosage escalation steps, wherein the dosage in each of the dosage escalation steps is in an amount of up to about 2.4 mg per week.

46. The method according to claim 45, wherein the dosage in each of the dosage escalation steps is about 2.4 mg per week.

47. The method according to claim 42, wherein the semaglutide is administered once weekly.

48. The method according to claim 47, wherein the human subject has obesity.

49. The method according to claim 47, wherein the human subject has overweight.

50. The method according to claim 47, wherein prior to the administering of the about 7.2 mg of semaglutide once weekly, administering semaglutide to the subject in one or more dosage escalation steps, wherein the dosage in each of the dosage escalation steps is in an amount of up to about 2.4 mg once weekly.

51. The method according to claim 50, wherein the dosage in each of the dosage escalation steps is about 2.4 mg once weekly.

52. The method according to claim 42, wherein the semaglutide is administered in an amount of 7.2 mg weekly.

53. The method according to claim 52, wherein the human subject has obesity.

54. The method according to claim 52, wherein the human subject has overweight.

55. The method according to claim 52, wherein prior to the administering of the 7.2 mg per week of semaglutide, administering semaglutide to the subject in one or more dosage escalation steps, wherein the dosage in each of the dosage escalation steps is in an amount of up to about 2.4 mg per week.

56. The method according to claim 55, wherein the dosage in each of the dosage escalation steps is about 2.4 mg per week.

57. The method according to claim 56, wherein the dosage in each of the dosage escalation steps is 2.4 mg per week.

58. The method according to claim 52, wherein the semaglutide is administered once weekly.

59. The method according to claim 58, wherein the human subject has obesity.

60. The method according to claim 58, wherein the human subject has overweight.

61. The method according to claim 58, wherein prior to the administering of the 7.2 mg of semaglutide once weekly, administering semaglutide to the subject in one or more dosage escalation steps, wherein the dosage in each of the dosage escalation steps is in an amount of up to about 2.4 mg once weekly.

62. The method according to claim 61, wherein the dosage in each of the dosage escalation steps is about 2.4 mg once weekly.

63. The method according to claim 62, wherein the dosage in each of the dosage escalation steps is 2.4 mg once weekly.
